(19) United States
(12) Patent Application Publication
(10) Pub. No.: US 2014/0088073 A1
(43) Pub. Date: Mar. 27, 2014

(54) METHODS FOR DETERMINING WHETHER A PATIENT SHOULD BE ADMINISTERED A DRUG THAT INHIBITS CHOLESTEROL ABSORPTION

(71) Applicant: Boston Heart Diagnostics Corporation, Framingham, MA (US)

(72) Inventor: Ernst J. Schaefer, Natick, MA (US)

(73) Assignee: BOSTON HEART DIAGNOSTICS CORPORATION, Framingham, MA (US)

(21) Appl. No.: 13/950,732

(22) Filed: Jul. 25, 2013

Related U.S. Application Data
(60) Provisional application No. 61/706,638, filed on Sep. 27, 2012.

(51) Int. Cl. A61K 31/397

(52) U.S. Cl. CPC A61K 31/397
USPC 514/210.02

(57) ABSTRACT

The invention generally relates to methods for determining whether a patient should be administered a drug that inhibits cholesterol absorption. In certain aspects, methods of the invention involve obtaining a sample from a patient, conducting an assay on the sample to obtain a level of a cholesterol absorption marker, and comparing the level to a reference level, in which a level above the reference level indicates that the patient should be administered a drug that inhibits cholesterol absorption.

### RELATED APPLICATION

This application claims the benefit of and priority to U.S. Provisional App. Ser. No. 61/706,638, filed Sep. 27, 2012, the entirety of which is incorporated by reference.

### FIELD OF THE INVENTION

The invention generally relates to methods for determining whether a patient should be administered a drug that inhibits cholesterol absorption.

### BACKGROUND

Cholesterol is an essential structural component of mammalian cell membranes and is required to establish proper membrane permeability and fluidity. In addition to its importance within cells, cholesterol also serves as a precursor for the biosynthesis of steroid hormones, bile acids, and vitamin D.

Although cholesterol is important and necessary for human health, high levels of cholesterol in the blood have been linked to damage to arteries and cardiovascular disease. To control risk factors, a patient's cholesterol is routinely monitored. Standard cholesterol screening tests are conducted by obtaining a blood sample from a patient and measuring a total cholesterol level, a low density lipoprotein cholesterol (LDL) level, and a high density lipoprotein cholesterol (HDL) level. The LDL cholesterol is also known as “bad cholesterol” because it promotes plaque formation on the inner walls of arteries. Together with other substances, LDL cholesterol is thought to cause atherosclerosis. If a clot forms and blocks a narrowed artery, heart attack or stroke can result.

HDL cholesterol is also known as “good cholesterol” because high levels of HDL are cardioprotective. It is believed that HDL tends to carry cholesterol away from the arteries and back to the liver for metabolism. Additionally, HDL removes excess cholesterol from arterial plaque, slowing its buildup. Generally, a patient should have a total cholesterol level of about 200 (HDL, LDL, and other lipoproteins), an LDL level less than 100, and an HDL level of 60 or above.

A patient's high cholesterol level may be caused by over-production of cholesterol in the body, excessive absorption of cholesterol by the body, or a combination of the two. Over-production of cholesterol results in a high total cholesterol level. Excessive absorption of cholesterol manifests as a high LDL level. Either a high total cholesterol level or a high LDL level may cause a patient to develop artery damage or cardiovascular disease. In fact, many patients with a normal total cholesterol level are at risk of developing artery damage or cardiovascular disease due to a high LDL level.

Changes to diet and level of physical activity can help lower cholesterol levels. Drugs may also be used to help lower cholesterol levels. Statins are generally administered to patients having high total cholesterol. Statins are a class of drugs used to lower cholesterol levels by inhibiting the enzyme HMG-CoA reductase, which plays a central role in the production of cholesterol in the liver. Thus, statins act by inhibiting cholesterol synthesis.

However, statins do not regulate cholesterol absorption, and in fact have been found to up regulate cholesterol absorption (van Himberhen, J Lipid Res, 50:730-739, 2009). Thus, statins are not effective at preventing the development of artery damage or cardiovascular disease in patients that have a high LDL level and may even exacerbate the problem in a certain population of patients by adversely affecting cholesterol absorption.

### SUMMARY

The invention recognizes that cholesterol adsorption is linked to a patient's low density lipoprotein level (LDL), and that monitoring for markers of cholesterol absorption indicates whether a patient would benefit from a drug that inhibits cholesterol absorption. For example, a patient with hypercholesterolemia may have elevated LDL-C despite being on a statin. A determination of levels of markers, such as sterols, associated with cholesterol absorption indicates whether an absorption inhibitor, such as ezetimibe, will reduce the elevated LDL-C. In addition, the invention is useful as an assay to monitor marker signatures, such as the relative amounts of different sterols or other molecules associated with cholesterol absorption, as the indicator of absorption inhibitor efficacy. Amounts or relative amounts of markers for cholesterol absorption are predictive of absorption inhibitor efficacy. Those amounts are determined by reference to standards or are determined empirically. The invention is also useful to monitor patients who have received a statin and/or an absorption inhibitor. Examples of use of the invention are provided below. Any drug that inhibits cholesterol absorption may be used with methods of the invention and a particularly useful drug is ezetimibe.

Generally, methods of the invention are conducted using a blood sample, however, any tissue or body fluid sample that includes markers for cholesterol absorption may be used with methods of the invention e.g., fecal or urine samples. Methods of the invention further involve conducting an assay on the sample to obtain a level of a cholesterol absorption marker. Any cholesterol absorption marker or combination of cholesterol absorption markers may be used with methods of the invention. In certain embodiments, the marker is a steroid alcohol (sterol), such as campesterol or β-sitosterol.

Methods of the invention are useful with any patients that are at risk of artery damage or developing a cardiovascular disease. Patient may have a high total cholesterol level, a high LDL level, or a combination thereof. The patient may or may not be already taking a statin. In certain embodiments, the patient is also taking a statin. The statin may be a low-potency statin, a medium potency statin, or a high-potency statin. In certain embodiments, the statin is a high-potency statin, such as simvastatin or atorvastatin.

Other aspects of the invention provide methods for treating a patient with high LDL-C that involve obtaining a sample from a patient, conducting an assay on the sample to obtain a level of a cholesterol absorption marker, comparing the level to a reference level, in which a level above the reference level indicates that the patient should be administered a drug that inhibits cholesterol absorption, and administering to the patient a drug that inhibits cholesterol absorption, thereby treating the patient with high LDL-C. In certain embodiments, a level of a cholesterol production marker is also examined and compared to a reference level in order to determine whether a drug that inhibits cholesterol absorption should be administered.

Other aspects of the invention provide methods for determining whether a patient taking a statin should additionally be administered a drug that inhibits cholesterol absorption. Those methods involve obtaining a sample from a patient that is taking a statin, conducting an assay on the sample to obtain a level of a cholesterol absorption marker, and comparing the level to a reference level, in which a level above the reference level indicates that the patient should additionally be administered a drug that inhibits cholesterol absorption. Methods of the invention may further involve administering the drug that inhibits cholesterol absorption, such as ezetimibe.

### BRIEF DESCRIPTION OF THE DRAWINGS

FIG. 1, Panels A and B depict baseline plasma sterol/cholesterol levels during on-going statin therapy. Panel A. baseline lathosterol/cholesterol, and Panel B. baseline b-sitosterol/cholesterol.

FIG. 2. depicts percent change in lipid values from statin-treated baseline after ezetimibe add-on therapy (adjusted for values from the placebo arm of EASE).

FIG. 3, Panels A, B, C, and D depict effect of 6-week ezetimibe add-on treatment on plasma non-cholesterol sterol levels as shown as absolute and percent change by statin potency and type.

### DETAILED DESCRIPTION

Methods of the invention can be used to determine whether a patient should be administered a drug that inhibits cholesterol absorptions in order to lower the patient's LDL cholesterol. In certain embodiments, methods for treating a patient with high LDL include obtaining a sample from a patient, conducting an assay on the sample to obtain a level of a cholesterol absorption marker, comparing the level of the cholesterol absorption marker of the patient to a reference level. Based on the comparison, the methods provides for administering to the patient a drug that inhibits cholesterol absorption, thereby treating the patient with high LDL.

Methods of the invention are used to treat heart disease associated with high LDL cholesterol. Heart disease includes but is not limited to coronary heart disease (CHD), cardiomyopathy, cardiovascular disease (CVD), ischemic heart disease, heart failure, hypertensive heart disease, inflammatory heart disease, and valvular heart disease. Heart disease is a systemic disease that can affect the heart, brain, most major organs, and the extremities. Coronary heart disease that causes the failure of coronary circulation to supply adequate circulation to the cardiac muscles and surrounding tissues. Cardiovascular disease includes any of a number of specific diseases that affect the heart itself and/or the blood vessel system, especially the myocardial tissue, as well as veins and arteries leading to and from the heart. For example, CVD may include, but is not limited to, acute coronary syndromes, arrhythmia, atherosclerosis, heart failure, myocardial infarction, neointimal hyperplasia, pulmonary hypertension, stroke, and/or valvular disease. CVD may be diagnosed by any of a variety of methods known in the art. For example, such methods may include assessing a subject for dyspnea, orthopnea, paroxysmal nocturnal dyspnea, claudication, angina, chest pain, which may present as any of a number of symptoms known in the art, such as exercise intolerance, edema, palpitations, faintness, loss of consciousness, and/or cough.

Atherosclerosis is a heart disease in which an artery wall thickens as the result of a build-up of fatty materials such as cholesterol. It is a syndrome affecting arterial blood vessels, a chronic inflammatory response in the walls of arteries, in large part due to the accumulation of macrophage white blood cells and promoted by low-density lipoproteins (plasma proteins that carry cholesterol and triglycerides) without adequate removal of fats and cholesterol from the macrophages by functional high density lipoproteins (HDL). It is commonly referred to as a hardening or furring of the arteries. It is caused by the formation of multiple plaques within the arteries.

Methods of the invention contemplate the use of patient-derived samples that are used in assays or tests in order to obtain particular information. Samples generally refer to biological samples isolated from a subject and can include, without limitation, whole blood, serum, plasma, blood cells, endothelial cells, tissue biopsies, lymphatic fluid, ascites fluid, interstitital fluid (also known as “extracellular fluid” and encompasses the fluid found in spaces between cells, including, inter alia, gingival crevicular fluid), bone marrow, cerebrospinal fluid (CSF), saliva, mucous, sputum, sweat, urine, or any other secretion, excretion, or other bodily fluids. In preferred embodiments, the patient sample is a blood sample, which can include whole blood or any fraction thereof, including blood cells, serum and plasma.

Methods of the invention may be used to improve a patient's total cholesterol levels, LDL cholesterol levels, HDL cholesterol levels, or combinations thereof. In certain embodiments, methods of the invention provide for administering a drug that inhibits cholesterol absorption to improve total cholesterol levels, LDL cholesterol levels, HDL cholesterol levels, or combinations thereof. The aim of therapy is to achieve cholesterol levels (total, LDL, HDL) that are normal or levels that are indicative of a lower risk of heart disease. Ideal guidelines for total cholesterol levels, LDL cholesterol levels, and HDL cholesterol levels are described hereinafter.

According to certain embodiments, methods of the invention are used to determine whether a drug that inhibits cholesterol absorption should be administered in order to lower density lipoprotein (LDL) cholesterol (LDL-C), which is associated with increased heart risk. The following are typical guidelines for LDL cholesterol levels. LDL cholesterol below 70 mg/dL (1.8 mmol/L) is ideal for people at very high risks of heart disease. LDL cholesterol below 100 mg/dL (2.6 mmol/L) is ideal for people at risk of heart disease and normal patient populations. LDL cholesterol levels between 100 mg/dL and 129 mg/dL (2.6-3.3 mmol/L) is near ideal. LDL cholesterol levels between 130 mg/dL and 159 mg/dL (3.4-4.1 mmol/L) is borderline high. LDL cholesterol levels between 160 mg/dL and 189 mg/dL (4.1-4.9 mmol/L) is high. LDL cholesterol levels between 190 mg/dL and above (above 4.9 mmol/L) is very high.

In certain embodiments, methods of the invention may be used to determine whether a drug that inhibits cholesterol absorption should be used to increase high density lipoprotein (HDL) cholesterol (HDL-C). HDL-C can protect against atherosclerosis in several ways. The most cited HDL-C function to protect against atherosclerosis is its participation in reverse cholesterol transport. During this process, HDL-C removes cholesterol from macrophages in the vessel wall, preventing the transformation of macrophages into foam cells, eventually preventing the build-up of fatty streaks and plaque in the vessel wall. HDL-C also acts as an anti-oxidant and anti-inflammatory agent, which prevents oxidation of LDL and reduces cholesterol build-up caused by oxidized LDL. The following are typical guidelines for HDL cholesterol levels. HDL cholesterol levels below 40 mg/dL (men) and below 50 mg/dl (women) is considered poor. HDL cholesterol levels between 40-49 mg/dL (men) and 50-59 mg/dL (women) is intermediate. HDL cholesterol levels above 60 mg/dL are ideal.

In addition, methods of the invention may be used to determine whether a drug that inhibits cholesterol absorption should be administered to lower total cholesterol. The following are typical guidelines for total cholesterol levels. Total cholesterol below 200 mg/dL is ideal/normal. Total cholesterol ranging between 200 and 239 mg/dL is borderline high for risk of heart disease. Total cholesterol above 240 mg/dL is high for risk of heart disease.

A key step for determining whether a patient should be administered a drug that inhibits cholesterol absorption is analyzing one or more cholesterol absorption biomarkers of the patient. Preferably, the one or more cholesterol markers are sterol markers. Sterol markers include cholesterol absorption markers, cholesterol production markers, or combinations thereof. Cholesterol absorption markers allow one to determine a level of cholesterol, received through the diet that is absorbed by the small intestine. Cholesterol production markers allow one to determine how much cholesterol cells are synthesizing.

An individual's ability to produce and absorb cholesterol is an important factor contributing the individual's total cholesterol and LDL cholesterol. This is because all LDL-C present in the body is the result of production of cholesterol from the liver and absorption of cholesterol from the diet. By analyzing a patient's cholesterol production and/or absorption markers, one is able to determine how the patient absorbs and absorbs cholesterol. Some people synthesize cholesterol more than they absorb cholesterol (over-producers), while others absorb more cholesterol than they synthesize (over-absorber).

Knowing how an individual produces and absorbs cholesterol allows one to determine and prescribe the most appropriate course of treatment, either at the initiation of initial therapy or at a change in current therapy, because each mechanism can be controlled by a different cholesterol lowering drug. For example, an over-producer will achieve lower cholesterol levels if prescribed a drug that inhibits cholesterol production Likewise, an over-absorber will achieve lower cholesterol levels if prescribed a drug that inhibits cholesterol absorption. Thus, prescribing a course of treatment directed towards a patient's cholesterol absorption or production markers allows one to reliably predict how a compliant patient will respond to the prescribed course of treatment.

Cholesterol production markers include, for example, lathosterol and desmosterol. About eighty percent of synthesized cholesterol goes through lathosterol, while about 20% of synthesized cholesterol goes through desmosterol. People who overproduce cholesterol have elevated levels of lathosterol and desmosterol normalized to total blood cholesterol levels. As a result, levels of lathosterol and desmosterol can be used as markers to determine whether an individual is an overproducer of cholesterol.

Cholesterol absorption markers include, for example, beta-sitosterol and campesterol. These plant sterols are direct measures of cholesterol absorption. Individuals who over-absorb cholesterol in the intestine have elevated levels of these markers. Decreased values, which reflect low cholesterol absorption, are optimal.

The following tables categorize optimal, borderline, high, and very high levels of cholesterol absorption markers and cholesterol production markers for men and women.

TABLE 4

| | Production Markers | | Absorption Markers | |
|---|---|---|---|---|
| Women | Lathosterol | Desmosterol | Beta-sitosterol | Campesterol |
| Optimal | <130 | <70 | <130 | <180 |
| Borderline | 130-150 | 70-80 | 130-160 | 180-300 |
| High | >150 | >80 | >250 | >400 |
| Very High | >200 | >150 | >250 | >400 |

(Sterol values in moles × 10² mol of cholesterol)

TABLE 5

| | Production Markers | | Absorption Markers | |
|---|---|---|---|---|
| Men | Lathosterol | Desmosterol | Beta-sitosterol | Campesterol |
| Optimal | <120 | <70 | <150 | <200 |
| Borderline | 120-135 | 70-75 | 150-160 | 200-220 |
| High | >135 | >75 | >160 | >300 |
| Very High | >200 | >150 | >250 | >400 |

(Sterol values in moles × 10² mol of cholesterol)

Certain aspects of the invention involve conducting an assay to determine a level of a patient's cholesterol production marker, cholesterol absorption marker, or both. From the assay, method of the invention provide for determining whether the patient is an over-absorber or an over-producer of cholesterol using the cholesterol production markers, the cholesterol absorption markers, or both, which are outlined in Tables 4 and 5. Whether a person is an overproducer or an overabsorber are important factors in determining a course of treatment for lowering cholesterol. Individuals who are overproducers of cholesterol benefit from a drug that inhibits cholesterol production (such as a statin). Individuals who are over-absorbers of cholesterol benefit from a drug that inhibits cholesterol absorption (such as an ezetimibe). This sterol analysis is important because it provides a guideline for a physician to prescribe a course of treatment best suited for the individual based on the sterols. In certain embodiments, high or very high levels of cholesterol absorption markers are indicative that a patient is an over-absorber and would benefit from a therapy to inhibit cholesterol absorption. In other embodiments, high or very high levels of cholesterol production markers are indicative that a patient is an over-producer and would benefit from a therapy to inhibit cholesterol production.

According to certain embodiments, methods of the invention provide for balancing the cholesterol production markers against the cholesterol absorption markers to determine whether the patient is an over-absorber or over-producer. In these embodiments, an assay is conducted to determine levels of one or more cholesterol production markers and an assay is conducted to determine one or more cholesterol production markers of an individual. The assay of the cholesterol production markers may be the same or different from the assay for the cholesterol absorption markers. The cholesterol production marker levels are then compared against the cholesterol absorption marker levels. This comparison step allows one to determine whether it is the amount of cholesterol produced by the body or the amount of cholesterol absorb by the intestine that is contributing to total blood cholesterol and LDL cholesterol levels.

In certain embodiments, methods of the invention provide for assigning a weighted value to each risk category (optimal, borderline, high, very high) for each cholesterol absorption marker and each cholesterol production marker. A weighted value for cholesterol production markers may be compare to a weighted value for cholesterol absorption markers to determine whether an individual is an over-producer or over-absorber. The weighted value may be scaled in any manner including and not limited to assigning a positive or negative integer to reflect the significance or severity of the risk category towards increasing cholesterol. The weighted value for each risk category for each marker may also take into consideration the percent contribution (n) that marker has towards the patient's cholesterol levels. For example, lathosterol contributes to 80% of synthesized cholesterol and desmosterol contributes to only 20% of synthesized cholesterol. The weighted values of the cholesterol absorption markers can be compared to the weighted values of the cholesterol production markers to determine which one is contributing more to the cholesterol. For example, Table 6 shows a simplified method for assigning weighted values to cholesterol production and cholesterol absorption markers for men. While weighted values of Table 6 are shown for illustrative purposes, any method for quantitatively and qualitatively comparing the production markers to the absorption markers may be used. It is also understood that the same concept may apply to those markers for women in Table 5.

TABLE 6

| | | | Absorption Markers | |
|---|---|---|---|---|
| | Production Markers | | Beta- | |
| Men | Lathosterol | Desmosterol | sitosterol | Campesterol |
| Optimal | <120 | <70 | <150 | <200 |
| | Weighted | Weighted | Weighted | Weighted Value: |
| | Value: 1 * n | value: 1 * n | Value: | −1 * n |
| | | | −1 * n | |
| Borderline | 120-135 | 70-75 | 150-160 | 200-220 |
| | Weighted | Weighted | Weighted | Weighted Value: |
| | Value: 2 * n | Value: 2 * n | Value: | −2 * n |
| | | | −2 * n | |
| High | >135 | >75 | >160 | >300 |
| | Weighted | Weighted | Weighted | Weighted Value: |
| | Value: 3 * n | Value: 3 * n | Value: | −3 * n |
| | | | −3 * n | |
| Very High | >200 | >150 | >250 | >400 |
| | Weighted | Weighted | Weighted | Weighted Value: |
| | Value: 4 * n | Value: 4 * n | Value: | −4 * n |
| | | | −4 * n | |

As shown in Table 6, each category for each biomarker is assigned a weighted value that is multiplied by that markers contribution to either cholesterol production or cholesterol absorption. Using the weighted values, one can balance the cholesterol absorption values against the cholesterol production values. The following are non-limiting guidelines for balancing cholesterol production with cholesterol absorption. When the sum of cholesterol absorption and cholesterol production values totals −1, 0, or 1, the individual is producing cholesterol at a substantially similar level as the individual is absorbing cholesterol. As a result, the individual is classified as a balanced producer. When the sum of the cholesterol absorption and cholesterol production values is less than −1, the individual is over-absorbing cholesterol and is classified as an over-absorber. When the sum of the cholesterol absorption and cholesterol production values is greater than 1, the individual is over-producing cholesterol and is classified as an over-absorber.

Once an individual is established as an over-producer, a balanced producer, or an over-absorber, methods of the invention provide for prescribing/administering a course of treatment designed to lower the cholesterol of the patient. For balanced producers, the individual may be prescribed a cholesterol production inhibiting drug alone or in combination with an ezetimibe in order to reduce cholesterol. For over-producers, the individual may be prescribed a cholesterol production inhibiting drug. As such, methods of the invention provide for administering a drug that inhibits cholesterol production (such as a high potency statin) if the individual is an over producer. For over-absorbers, the individual may be prescribed a cholesterol absorption inhibiting drug. As such, methods of the invention provide for administering a drug that inhibits cholesterol absorption, such as an ezetimibe) if the cholesterol balancing test indicates that the individual is an over-absorber.

In some instances, a patient may already be on a cholesterol reducing therapy. In such instances, the cholesterol balance test can be utilized to provide a course of treatment to enhance the current cholesterol reducing therapy. For example, balancing the cholesterol production makers and cholesterol absorption makers of a patient already undergoing a statin therapy may show that the patient is an over-absorber of cholesterol. This is because the statin therapy while decreasing the cholesterol production markers also increased the cholesterol absorption markers. In this example, the course of treatment may be to add a cholesterol absorption inhibiting drug to the statin therapy in order to assist in decreasing cholesterol. The benefits of combining cholesterol absorption inhibiting drugs to a cholesterol production inhibiting therapy are described in more detail in Thongtang et al., “Effects of ezetimibe added to statin therapy on markers of cholesterol absorption and synthesis and LDL-C lowering in hyperlipidemic patients,” Atherosclerosis, Volume 225, Issue 2, December 2012, Pages 388-396, the entirety of which is incorporated by reference.

In addition, the degree to which an individual is classified as an over-producer or an under-producer can be used to determine, for example, the dosage and type of drug the patient should be administered. For example, if the cholesterol balancing test indicates that high risk levels of cholesterol production markers are responsible for elevated total cholesterol (e.g. when there is minimal to no levels of cholesterol absorption markers), then a high statin dosage and/or a medium to high potency statin may be appropriate. In another example, if the cholesterol balancing test indicates that the patient is a balanced producer, yet still has high cholesterol, the patient may benefit from a low or medium potency statin along with ezetimibe treatment.

The cholesterol production inhibiting drug is typically a statin. A statin is class of drugs used to lower cholesterol levels by inhibiting the enzyme HMG-CoA reductase, which plays a central role in the production of cholesterol in the liver. Statins may include but are not limited to Advicor® (niacin extended-release/lovastatin), Altoprev® (lovastatin extended-release), Caduet® (amlodipine and atorvastatin), Crestor® (rosuvastatin), Lescol® (fluvastatin), Lescol XL (fluvastatin extended-release), Lipitor® (atorvastatin), Livalo® (pitavastatin), Mevacor® (lovastatin), Pravachol® (pravastatin), Simcor® (niacin extended-release/simvastatin), Vytorin® (ezetimibe/simvastatin), Zocor® (simvastatin), or generic atorvastatin, lovastatin, pravastatin, or simvastatin. Typically, statin dosage is the amount of a statin required to reduce LDL-C to target level relative to an untreated patient. Statin dosage may depend upon the manner of administration, the age, body weight, and general health of the subject. Additionally, statin dosage may vary depending upon which statin is being administered. For example, a typical statin dosage range for atorvastatin, pravastatin, lovastatin, fluvastatin and simvastatin is from about 10 mg to about 80 mg. Low potency statins are predicted at lowering LDL-C by ˜20-30%, and may include the following stains and dosages: simvastatin ≦10 mg/day, lovastatin ≦20 mg/day, pravastatin ≦20 mg/day, and fluvastatin ≦40 mg/day. The medium potency statins are predicted at lowering LDL-C by ˜31-45%, and may include the following statins and dosages: simvastatin >10 to ≦40 mg/day, atorvastatin ≦20 mg/day, lovastatin >20 to 80 mg/day, pravastatin >20 to 80 mg/day, and fluvastatin >40 to 80 mg/day. The high potency statins are predicted at lowering LDL-C by ˜46-55%, and may include the following statins and dosages: simvastatin >40 to 80 mg/day, and atorvastatin >20 to 80 mg/day.

Drugs that inhibit cholesterol absorption that are suitable for use in methods of the invention include the drug ezetimibe, candicidin and other polyene macrolides, or bile acid sequestering anionic exchange resins such as Cholestyramine® and Colestipol®. Preferably, the cholesterol absorption drug is ezetimibe. Ezetimibe's mode of action involves the inhibition of cholesterol absorption and resorption in the intestinal tract. This mechanism of action also involves the increased excretions of cholesterol and its intestinal generated metabolites with the feces. This effect of ezetimibe results in lowered body cholesterol levels, increased cholesterol synthesis, and decreased triglyceride synthesis. The increased cholesterol synthesis initially provides for the maintenance of cholesterol levels in the circulation, levels that eventually decline as the inhibition of cholesterol absorption and resorption continues. The overall effect of drug action is the lowering of cholesterol levels in the circulation and tissues of the body. Ezetimibe is typically delivered n 10 mg/day dosages, when used alone or in combination with a statin therapy.

By establishing an individual as an over-producer, a balanced producer, or an over-producer using cholesterol absorption and cholesterol production markers, one can reliably predict how a compliant individual will respond to the prescribed treatment. For example, an over-producer of cholesterol on a statin therapy should achieve lower cholesterol when compliant with the statin therapy. An over-absorber of cholesterol on an ezetimibe therapy should achieve lower cholesterol when compliant with the ezetimibe therapy. A balanced producer on a statin therapy or a combined statin and ezetimibe therapy should achieve lower cholesterol when compliant with either therapy.

Methods of the invention provide a physician with the ability to predict how a patient will respond to a course of treatment, which allows the physician to determine whether the patient is complying with the prescribed cholesterol lowering therapy. In certain aspects, methods of the invention provide for monitoring/determining an actual change in a patient's cholesterol level in order to determine whether the patient is appropriately responding with the course of treatment. In certain embodiments, an assay is conducted to determine a level of cholesterol from a sample of a patient undergoing the course of treatment. The sample level of cholesterol is then compared to a reference level of cholesterol. Ideally, the patient is undergoing the course of treatment for a period of time after the reference level is obtained. The period of time should be long enough for the course of treatment to have an effect on the patient's cholesterol level. The reference level of cholesterol may be the patient's cholesterol level prior to the start of the initial treatment or prior to the initiation of a different treatment (such as an increased dosage or different drug type). If the individual's cholesterol lowers as predicted for the course of treatment, the patient is classified as compliantly taking the prescribed course of treatment. If the individual's cholesterol remains substantially unchanged (substantially zero change) or increases, the patient is classified as non-compliant.

As an alternative to monitoring cholesterol levels or in conjunction with monitoring cholesterol levels, an individual's cholesterol biomarker levels may be monitored to determine whether a patient is compliant with treatment. For example, an over-producer of cholesterol on a statin therapy should achieve lower levels of cholesterol production markers. In another example, an over-absorber of cholesterol on an ezetimibe therapy should achieve lower levels of cholesterol absorption biomarkers when compliant with the ezetimibe therapy.

In certain aspects, methods of the invention provide for monitoring an actual change in a patient's cholesterol markers in order to determine whether the patient is complying with statin therapy. In certain embodiments, a sample level of one or more cholesterol markers is obtained from a patient undergoing a statin therapy. The sample level of one or more cholesterol markers is then compared to a reference level of cholesterol markers. The reference level of one or more cholesterol markers may be the patient's level of cholesterol markers prior to initiation of statin therapy, the patient's level of cholesterol markers on a different statin therapy (e.g. lower dose or different cholesterol lowering drug), or known, typically cholesterol marker levels from a patient reference population (e.g. patient population with similar attributes as the patient). Ideally, the patient is undergoing the course of treatment for a period of time after the reference level is obtained. The period of time should be long enough for the course of treatment to have an effect on the patient's cholesterol level. If the individual's level of a cholesterol biomarker lowers as predicted for compliancy, the patient is classified as compliant. If the individual's level of cholesterol biomarker remains substantially unchanged (substantially zero change) or increases, the patient is classified as non-compliant.

By identifying non-compliance, the provider can confront the individual with the evidence of non-compliance and identify the cause of the patient's non-compliance. In addition, the healthcare provider is able to administer the appropriate drugs in order to assist the patient in efficiently reaching his LDL-C goals. For example, the provider may administer/prescribe the same course of treatment with a better plan for assisting the patient to remain compliant. In another example, if the non-compliance is due to the patient's side effects on the course of treatment, the provider may prescribe an alternative therapy that has the same cholesterol lowering benefits without the side effects or the provider may prescribe additional medication that reduces the side effects of the current course of treatment.

Additionally, if the patient is found compliant, the provider is able to objectively determine the next step of treatment because the provider no longer has to rely on patient's self-reporting for compliance. If the compliant, over-producer patient's cholesterol levels are still in a high risk category, the doctor may, for example, increase the statin dosage. If the compliant, over-absorber patient's cholesterol levels are still in a high risk category, the doctor may, for example, provide a different diet plan with the ezetimibe treatment in an effort to achieve cholesterol lowering goals.

Any assay for measuring cholesterol may be used in accordance with methods of the invention. The assay may be to determine a level of total cholesterol, HDL cholesterol, or LDL cholesterol. Rifai et al., Handbook of Lipoprotein Testing (Amer. Assoc. for Clinical Chemistry, 2000) provides a general outline of various assays for measuring total cholesterol, HDL cholesterol, and LDL cholesterol.

In one embodiment, a Liebermann-Burchard (L-B) assay is used to measure total cholesterol in blood. This is an absorbance-based assay. First, the L-B reaction reagent is prepared, which consists of solution of 30% glacial acetic acid, 60% acetic anhydride, and 10% sulphuric acid. Secondly, 5 ml of this L-B reagent is then added to 0.2 ml of a sample derived from blood plasma, which are mixed together and then allowed to stand for 20 minutes. The L-B reaction is usually carried out on a sample comprising cholesterol that has been extracted from plasma into an organic solvent. The products of the L-B reaction are two colored products. The absorbance of the products, the concentration of which are related to the concentration of cholesterol, is then measured using a spectrophotometer. The total concentration of cholesterol may be determined from a calibration curve of absorbance against cholesterol concentration, using cholesterol standards (Burke et ah, Clin. Chem. 20(7), 794-801 (1974)). Total cholesterol can also be measured using an isotope dilution-mass spectrometric method, which is described in Schaffer, R., et al. “Comparison of two isotope dilution/mass spectrometric methods for determination of total serum cholesterol.” Clinical chemistry 28.1 (1982): 5-8.

In another example, an enzymatic method is used for the determination of total cholesterol. In these methods, free cholesterol and esterified cholesterol are subjected to chemical or enzymatic saponification to convert the latter cholesterol to free cholesterol. All free cholesterols are allowed to interact with a cholesterol oxidase, and the formed hydrogen peroxide, cholestenone, or consumed oxygen is measured. See Clin. Chem., 20, 470, 1974; U.S. Pat. Nos. 3,925,164 and 4,212,938). These formed products are used as a measure of cholesterol. In some methods, the formed hydrogen peroxide is allowed to react with a peroxides and a color-producing reagent. The resulting colored substance is used as a measure of total cholesterol. Alternatively, a cholesterol dehydrogenase and NAD or NADP as a coenzyme are used can be used to interact with the free cholesterol to form cholestenone or the reduced type NAD (after referred to as NADH) or reduced type NADP (after referred to as NADPH). (U.S. Pat. No. 4,181,575; FRG Patent Laid-open No. 3,032,377 and Japanese Patent Laid-open No. 89,200/1983). These formed products are used as a measure of total cholesterol.

Any assay for measuring LDL cholesterol may be used in accordance with methods of the invention. A common approach for measuring LDL cholesterol is the Friedewald calculation method, which estimates LDL-C from measurements of total cholesterol, triglycerides, and HDL-cholesterol. Other approaches involve direct measurement of LDL cholesterol. For example, LDL cholesterol may be measured using ultracentrifugation methods, electrophoresis methods, precipitation methods, methods that use polyethylene-glycol modified enzymes, methods that use synthetic polymers, immunological separation methods, and catalase reagent methods.

Ultracentrifugation for measuring LDL cholesterol separates lipoproteins based on their differing hydrated densities, which are adjusted by adding salts such as NaBr or KBr. Particularly, the proportion of lipid associated with the proteins for any one particular lipoprotein adds to the buoyancy of the lipoprotein complex, which allows it to be separated. Preparative fractionations are achieved by subjecting serum or plasma to ultracentrifugation at the native non-protein solute density, which floats TG-rich BLDL and chylomicrons. Those can be recovered using tube slicing or aspiration. The bottom fraction contains the LDL and HDL, which can be re-centrifuged, after adding salt, to float LDL.

For measuring of LDL-C by electrophoresis, lipoproteins may be separated using a variety of electrophoric media, such as paper, agarose gel, cellulose acetate, and polyacrylamide with one or more buffers. A preferred electrophoresis separation and immune-detection technique is described in co-owned and co-assigned U.S. patent application Ser. No. 12/567,737. A common technique uses agarose gels to separate lipoproteins followed by precipitation with polyanions and densitometric scanning. This technique can be approved by the introduction of enzymatic cholesterol determination using cholesterol esterase, cholesterol dehydrogenase, and nitroblue tetrazolium chloride dye. An alternative technique uses agarose gel modified by addition of a cation such as magnesium, which slows migration of β and pre-β lipoproteins, producing a distinct additional band between pre-β and α lipoproteins, demonstrated to be Lp(a) by immunofixation. Addition of urea to the gel allowed simultaneous quantification of the β, pre-β, and α fractions as well as Lp(a)-cholesterol with the mobility of Lp(a) independent of apo(a) size polymorphisms.

Another technique for direct measurement of LDL cholesterol is an immunoseparation method, known as Direct LDL from Genzyme Diagnostics and Signma Diagnostics. This technique uses a reagent that contained polyclonal (goat) antibodies to human apo A-I m and apo E bound to polystyrene latex beads and that was designed to remove chylomicrons, HDL, VLDL, and IDL particles, allowing direct determination of LDL-C. In yet another technique for direct measurement of LDL cholesterol involves magnetic precipitation, such as a technique used by Reference Diagnostics. The magnetic precipitation technique used heparin-coated beads at pH 5.1 to remove LDL from serum, leaving HDL and VLDL remaining in the solution.

In addition, homogenous assays for measuring LDL cholesterol may be used. One homogeneous method for determining LDL-C is disclosed in U.S. Pat. No. 5,888,827 (Kayahara, Sugiuchi, et al.; assigned to Kyowa Medex Co., Japan). The '827 patent describes a two-stage liquid phase reaction to quantify LDL-C concentration in a fluid sample. In the first step, the sample containing LDL-C is placed in a first reagent that includes trimethyl beta-cyclodextrin as a sugar compound, polyoxyethylene monolaurate as a protein solubilizing agent, EMSE (N-ethyl-N-(3-methylphenyl)-N′,succinylethylenediamene) and Tris buffer. The reaction mixture is then heated to 37° C., and after 5 minutes the absorbance is read. A second reagent including cholesterol esterase, cholesterol oxidase, peroxidase, 4-aminoantipyrine and Tris buffer is then added and after another 5 minutes the absorbance is again measured at the same wavelength. LDL-C is then calculated by separately subjecting a standard solution of cholesterol to the same procedure and comparing the respective absorbance values.

Another two-stage homogeneous assay is disclosed in U.S. Pat. No. 6,194,164 (Matsui et al.; assigned to Denke Seiken, Ltd. Japan). In the first stage, HDL-C, VLDL-C and Chylomicron-C in the test sample are eliminated and, in the second step, the cholesterol remaining in the test sample (viz., LDL) is quantified. In the first step, cholesterol esterase and cholesterol oxidase act on the test sample in the presence of a surfactant that acts on lipoproteins other than LDL-C (“non-LDLs”). The hydrogen peroxide thereby generated is decomposed to water and oxygen by catalase. Alternatively, a phenol-based or aniline-based hydrogen donor is reacted with the hydrogen peroxide to convert it to a colorless compound. Preferred surfactants that act on the non-LDLs include polyoxyethylene laurl ether, polyoxyethylene cetyl ether, polyoxyethylene oleyl ether, polyoxyethylene higher alcohol ether, and the like. In the second reaction disclosed in the '164 patent, cholesterol remaining in the test sample, which should theoretically contain only LDL-C, is quantified. The second step may be carried out by adding a surfactant that acts on at least LDL and quantifying the resulting hydrogen peroxide by the action of the cholesterol esterase and the cholesterol oxidase added in the first step.

A homogeneous assay for measuring LDL-C in serum was disclosed by H. Sugiuchi et al., Clinical Chemistry 44:3 522-531 (1998). This disclosure shows a correlation between the use of a combination of triblock copolymer and alpha-cyclodextrin sulfate and the selective enzymatic reaction of LDL-C when both LDLs and non-LDLs are contacted with the combination in a liquid assay system.

In a homogenous assay from Diiachi Pure Chemicals Company, a3-μL serum sample is incubated with 300 μL of reagent 1 for 5 min at 37° C. Reagent 1 contains ascorbic acid, oxidase, 4-aminoantipyrene, peroxidase, cholesterol oxidase, cholesterol esterase, buffer (pH 6.3), and a detergent, which solubilizes all non-LDL lipoproteins. The cholesterol reacts with cholesterol esterase and cholesterol oxidase, generating hydrogen peroxide, which is consumed by a peroxidase in the presence of 4-aminoantipyrene with no color generation. Reagent 2 (100 μL) is then added, which contains N,N-bis-(4-sulfobutyl)-m-toluidine disodium salt, buffer (pH 6.3), and a specific detergent, which specifically releases cholesterol from LDL particles. An enzymatic reaction similar to that described above occurs except that the hydrogen peroxide reacts with N,N′-bis-(4-sulfobutyl)-m-toluidine disodium salt to generate a colored product [measured at 546 (main) and 660 (subsidiary) nm] that is proportional to LDL-C. A homogeneous LDL-C assay from International Reagents Corporation uses 5 μL of serum and 180 μL of reagent 1 with incubation. Calixarene, a detergent, converts LDL to a soluble complex. Cholesterol esters of HDL-C and VLDL-C are preferentially hydrolyzed by a cholesterol esterase (Chromobacterium); cholesterol oxidase and hydrazine then convert the accessible cholesterol to cholestenone hydrazone. In a second step, 60 μL of reagent 2 (deoxycholate) is added, breaking up the LDL-calixarene complex and allowing LDL-C to react with the esterase, a dehydrogenase, and β-NAD to yield cholestenone and β-NADH; the latter is measured spectrophotometrically.

Other assays for measuring LDL cholesterol include a solubilization LDL-C assay (SOL; Kyowa Medex), a surfactant LDL-C assay (SUR; Daiichi), a protecting reagent assay (PRO; Wako), a catalase LDL-C assay (CAT; Denka Seiken), and a calixarene LDL-C assay (CAL; International Reagents Corporation). Each of these assays are described in more detail in Nauck et al., Clinical Chemistry February 2002 vol. 48 no. 2 236-254.

Any assay for measuring HDL cholesterol may be used in accordance with methods of the invention. The general techniques for quantifying levels of HDL cholesterol are similar to and often the same techniques for quantifying levels of LDL cholesterol. For example, HDL cholesterol may also be measured using ultracentrifugation methods, electrophoresis methods, precipitation methods, methods that use polyethylene-glycol modified enzymes, methods that use synthetic polymers, immunological separation methods, and catalase reagent methods. These techniques and more are described in more detail in Warnick et al., Clinical Chemistry September 2001 vol. 47 no. 9 1579-1596.

Generally, ultracentrifugation techniques for measuring HDL-C separate lipoproteins based on their differing hydrated densities. Particularly, the proportion of lipid associated with the proteins for any one particular lipoprotein adds to the buoyancy of the lipoprotein complex, which allows it to be separated. This allows HDL-C to be separated from LDL-C, etc. For measuring HDL-C by electrophoresis, lipoproteins may be separated using a variety of electrophoric media, such as paper, agarose gel, cellulose acetate, and polyacrylamide with one or more buffers. A preferred electrophoresis separation and immune-detection technique is described in co-owned and co-assigned U.S. patent application Ser. No. 12/567,737. Lipoproteins separated by electrophoresis can be identified using immuno-detection techniques.

Precipitation and homogenous assays for separating HDL-C typically involve addition of two or more reagents to a sample, with incubation periods after addition of the reagents, followed by a measurement step, e.g. by colorimetric development or by UV/Vis analysis. For example, precipitation techniques for separating HDL-C involve the reaction of a precipitation reagent with low density lipoproteins (LDL), very low density lipoproteins (VLDL) and chylomicrons (CM) in order to form an aggregate of these components. The aggregate was then removed from the reaction vessel, for example by centrifugation, leaving an HDL-containing sample ready for analysis. Separation of the precipitate was essential in order that the precipitate did not interfere with the UV/Vis or colorimetric analysis techniques used. Homogenous assays for separating HDL particles are similar to precipitation assays, but typically do not require separation of precipitated lipoproteins. Instead, a clearing reagent is added to dissolve the precipitate after reaction with HDL-cholesterol is completed. In this way, the LDL, VLDL and CM are blocked ensuring selective reaction with HDL-cholesterol, but are cleared prior to carrying out the UV/Vis analysis. Alternatively, specific reaction conditions such as high dilution, or specific precipitation reagents, are used to ensure minimum interference with the analysis technique.

In certain embodiments, plasma total cholesterol (total C), high-density lipoprotein cholesterol (HDL-C), and triglycerides (TG) were analyzed using standardized methods at the central laboratory of the trial (PPD Global Central Labs, Highland Heights, Ky., USA). LDL-C was calculated using the Friedewald formula. Friedewald W T, Levy R I, Fredrickson D S. Estimation of the concentration of low-density lipoprotein cholesterol in plasma, without use of the preparative ultracentrifuge. Clin Chem 1972; 18:499-502. Non-HDL-C was calculated by subtracting HDL-C from total C.

Methods of the invention also utilize one or more assays in order to determine a level of one or more cholesterol absorption markers and to determine a level of one or more cholesterol production markers. The one or more assays to determine a level of one or more cholesterol absorption markers may be the same or different. The one or more assays to determine a level of one or more cholesterol production markers may be the same or different. In addition, the assay used to determine a level of cholesterol production marker may be the same or different.

Levels of cholesterol production markers and levels of cholesterol absorption markers can be determined using any assay known in the art. These biomarkers may readily be isolated and/or quantified by methods known to those of skill in the art, including, but not limited to, methods utilizing: mass spectrometry (MS), high performance liquid chromatography (HPLC), isocratic HPLC, gradient HPLC, normal phase chromatography, reverse phase HPLC, size exclusion chromatography, ion exchange chromatography, capillary electrophoresis, microfluidics, chromatography, gas chromatography (GC), thin-layer chromatography (TLC), immobilized metal ion affinity chromatography (IMAC), affinity chromatography, immunoassays, and/or colorimetric assays. In certain embodiments, cholesterol production and absorption markers are determined by using gas chromatography techniques, gas chromatography mass spectrometry (GC-MS) techniques, and/or high performance liquid chromatography (HPLC) techniques.

Gas chromatography techniques generally involve sample preparation, derivatization, and gas chromatography analysis. Sample preparation involves sample weighing, an optional step of lipid extraction, addition of an internal standard, hydrolysis (acid and/or alkaline), extraction of unsaponifiables, and purification. Derivation involves converting the substance (sterols) to be analyzed into a more volatile derivative that can be used for gas chromatography analysis. A gas chromatograph uses a flow-through narrow tube known as the column, through which different chemical constituents of a sample pass in a gas stream (carrier gas, mobile phase) at different rates depending on their various chemical and physical properties and their interaction with a specific column filling, called the stationary phase. As the chemicals exit the end of the column, they are detected and identified electronically. The function of the stationary phase in the column is to separate different components, causing each one to exit the column at a different time (retention time). Other parameters that can be used to alter the order or time of retention are the carrier gas flow rate, column length and the temperature. In a GC analysis, a known volume of gaseous or liquid analyte is injected into the “entrance” (head) of the column, usually using a microsyringe (or, solid phase microextraction fibers, or a gas source switching system). As the carrier gas sweeps the analyte molecules through the column, this motion is inhibited by the adsorption of the analyte molecules either onto the column walls or onto packing materials in the column. The rate at which the molecules progress along the column depends on the strength of adsorption, which in turn depends on the type of molecule and on the stationary phase materials. Since each type of molecule has a different rate of progression, the various components of the analyte mixture are separated as they progress along the column and reach the end of the column at different times (retention time). A detector is used to monitor the outlet stream from the column; thus, the time at which each component reaches the outlet and the amount of that component can be determined. Generally, substances are identified (qualitatively) by the order in which they emerge (elute) from the column and by the retention time of the analyte in the column.

Gas chromatography-mass spectrometry (GC-MS) is a method that combines the features of gas-liquid chromatography and mass spectrometry to identify different substances within a test sample. The GC-MS includes two components the gas chromatograph and the mass spectrometer. The gas chromatograph utilizes a capillary column which depends on the column's dimensions (length, diameter, film thickness) as well as the phase properties. The difference in the chemical properties between different molecules in a mixture will separate the molecules as the sample travels the length of the column. The molecules are retained by the column and then elute (come off) from the column at different times (called the retention time), and this allows the mass spectrometer downstream to capture, ionize, accelerate, deflect, and detect the ionized molecules separately. The mass spectrometer does this by breaking each molecule into ionized fragments and detecting these fragments using their mass to charge ratio. The most common type of mass spectrometer (MS) associated with a gas chromatograph (GC) is the quadrupole mass spectrometer, sometimes referred to by the Hewlett-Packard (now Agilent) trade name “Mass Selective Detector” (MSD).

Another relatively common detector is the ion trap mass spectrometer. Other detectors may be encountered such as time of flight (TOF), tandem quadrupoles (MS-MS), or in the case of an ion trap MSn where n indicates the number mass spectrometry stages. When a second phase of mass fragmentation is added, for example using a second quadrupole in a quadrupole instrument, it is called tandem MS (MS/MS). MS/MS can sometimes be used to quantitate low levels of target compounds in the presence of a high sample matrix background. The first quadrupole (Q1) is connected with a collision cell (q2) and another quadrupole (Q3). Both quadrupoles can be used in scanning or static mode, depending on the type of MS/MS analysis being performed. Types of analysis include product ion scan, precursor ion scan, selected reaction monitoring (SRM) (sometimes referred to as multiple reaction monitoring (MRM)) and neutral loss scan. For example: When Q1 is in static mode (looking at one mass only as in SIM), and Q3 is in scanning mode, one obtains a so-called product ion spectrum (also called “daughter spectrum”). From this spectrum, one can select a prominent product ion which can be the product ion for the chosen precursor ion. The pair is called a “transition” and forms the basis for SRM. SRM is highly specific and virtually eliminates matrix background.

In other embodiments, levels of cholesterol absorption markers and cholesterol production markers are determined using high-performance liquid chromatography (HPLC). HPLC relies on pumps to pass a pressurized liquid and a sample mixture through a column filled with a sorbent, leading to the separation of the sample components. The active component of the column, the sorbent, is typically a granular material made of solid particles (e.g. silica, polymers, etc.), 2-50 micrometers in size. The components of the sample mixture are separated from each other due to their different degrees of interaction with the sorbent particles. The pressurized liquid is typically a mixture of solvents (e.g. water, acetonitrile and/or methanol) and is referred to as “mobile phase”. Its composition and temperature plays a major role in the separation process by influencing the interactions taking place between sample components and sorbent. These interactions are physical in nature, such as hydrophobic (dispersive), dipole-dipole and ionic, most often a combination thereof. The schematic of an HPLC instrument typically includes a sampler, pumps, and a detector. The sampler brings the sample mixture into the mobile phase stream which carries it into the column. The pumps deliver the desired flow and composition of the mobile phase through the column. The detector generates a signal proportional to the amount of sample component emerging from the column, hence allowing for quantitative analysis of the sample components. A digital microprocessor and user software control the HPLC instrument and provide data analysis. Some models of mechanical pumps in a HPLC instrument can mix multiple solvents together in ratios changing in time, generating a composition gradient in the mobile phase. Various detectors are in common use, such as UV/Vis, photodiode array (PDA) or based on mass spectrometry. Most HPLC instruments also have a column oven that allows for adjusting the temperature the separation is performed at.

In addition, the following publications outline methods for measuring levels of cholesterol absorption and production biomarkers: Sudhop T, Lutjohann D, Kodal A, et al. “Inhibition of intestinal cholesterol absorption by ezetimibe in humans,” Circulation 2002; 106:1943-8; Matthan N R, Giovanni A, Schaefer E J, Brown B G, Lichtenstein A H., “Impact of simvastatin, niacin, and/or antioxidants on cholesterol metabolism in CAD patients with low HDL.” J Lipid Res. 2003; 44:800-806; Matthan N R, Raeini-Sarjaz M, Lichtenstein A H, Ausman L M, Jones P J., “Deuterium uptake and plasma cholesterol precursor levels correspond as methods for measurement of endogenous cholesterol synthesis in hypercholesterolemic women.”, Lipids. 2000; 35:1037-1044; Grundy, Scott. “Plasma Non-Cholesterol Sterols as Indicators of Cholesterol Absorption.” Journal of lipid research (2013); Tyburczy, Cynthia, et al. “Evaluation of low trans fat edible oils by attenuated total reflection-Fourier transform infrared spectroscopy and gas chromatography: a comparison of analytical approaches.” Analytical and bioanalytical chemistry 404.3 (2012): 809-819; Luzón-Toro, Berta, Alberto Zafra-Gómez, and Oscar Ballesteros. “Gas chromatographic-mass spectrometric determination of brain levels of α-cholest-8-en-3β-ol (lathosterol).” Journal of Chromatography B 850.1 (2007): 177-182; Mark, L., and G. Paragh. “[Change in the cholesterol metabolism associated with the combined inhibition of synthesis and absorption].” Orvosi hetilap 148.14 (2007): 627; Lund, E., et al. “Determination of serum levels of unesterified lathosterol by isotope dilution-mass spectrometry.” Scandinavian journal of clinical & laboratory investigation 49.2 (1989): 165-171; Goh, Edward H., Scott M. Colles, and Kimberly D. Otte. “HPLC analysis of desmosterol, 7-dehydrocholesterol, and cholesterol.” Lipids 24.7 (1989): 652-655.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### Example 1

Statins inhibit cholesterol synthesis but can upregulate cholesterol absorption, with higher doses producing larger effects. Ezetimibe inhibits cholesterol absorption which subsequently upregulates synthesis. Tests were performed to determine whether ezetimibe added to ongoing statin therapy would be most effective in lowering LDL-cholesterol (LDL-C) in subjects on high potency statins and whether these effects would be related to alterations in cholesterol absorption (β-sitosterol) and synthesis (lathosterol) markers.

Briefly, hypercholesterolemic subjects (n=874) on statins received ezetimibe 10 mg/day. Plasma lipids, lathosterol, and β-sitosterol were measured at baseline and on treatment. Subjects were divided into low (n=133), medium (n=582), and high (n=159) statin potency groups defined by predicted LDL-C lowering effects of each ongoing statin type and dose (reductions of ˜20-30%, ˜31-45%, or ˜46-55%, respectively).

The high potency group had significantly lower baseline lathosterol (1.93 vs. 2.58 vs. 3.17 μmol/l; p<0.001) and higher baseline β-sitosterol values (6.21 vs. 4.58 vs. 4.51 μmol/l, p<0.001) than medium/low potency groups. Ezetimibe treatment in the high potency group produced significantly greater reductions from baseline in LDL-C than medium/low potency groups (−29.1% vs. −25.0% vs. −22.7%; p<0.001) when evaluating unadjusted data. These effects and group differences were significantly (p<0.05) related to greater β-sitosterol reductions and smaller lathosterol increases. However LDL-C reduction differences between groups were no longer significant after controlling for placebo effects, due mainly to modest LDL-C lowering by placebo in the high potency group.

### Introduction

Statins play a central role in the treatment of atherogenic dyslipidemia and reduction of cardiovascular disease (CVD) risk. The cholesterol-lowering response to statin therapy, however, can vary widely between individuals. (See, e.g. Jones P H, Davidson M H, Stein E A, et al. Comparison of the efficacy and safety of rosuvastatin versus atorvastatin, simvastatin, and pravastatin across doses (STELLAR*Trial). Am J Cardiol 2003; 92:152-60; Weng T C, Yang Y H, Lin S J, Tai S H. A systematic review and meta-analysis on the therapeutic equivalence of statins. J Clin Pharm Ther 2010; 35:139-51). In addition, an individual's cholesterol lowering response to contributes to the substantial number of patients with LDL-C levels above guideline-recommended targets. (See, e.g. Third Report of the National Cholesterol Education Program (NCEP) Expert Panel on Detection, Evaluation, and Treatment of High Blood Cholesterol in Adults (Adult Treatment Panel III) final report. Circulation 2002; 106:3143-421; Grundy S M, Cleeman J I, Merz C N, et al. Implications of recent clinical trials for the National Cholesterol Education Program Adult Treatment Panel III guidelines. Circulation 2004; 110:227-39; Catapano A L, Reiner Z, De Backer G, et al. ESC/EAS Guidelines for the management of dyslipidaemias The Task Force for the management of dyslipidaemias of the European Society of Cardiology (ESC) and the European Atherosclerosis Society (EAS). Atherosclerosis 2011; 217:3-46; Perk J, De Backer G, Gohlke H, et al. European Guidelines on cardiovascular disease prevention in clinical practice (version 2012): The Fifth Joint Task Force of the European Society of Cardiology and Other Societies on Cardiovascular Disease Prevention in Clinical Practice (constituted by representatives of nine societies and by invited experts)*Developed with the special contribution of the European Association for Cardiovascular Prevention & Rehabilitation (EACPR). Eur Heart J 2012; 33:1635-701.)

Recent studies suggest that statin efficacy may be determined not only by their direct inhibitory effect on cholesterol synthesis but also by compensatory downstream changes in cholesterol metabolism. Statins reduce markers of cholesterol synthesis (e.g., lathosterol, desmosterol), which can elicit subsequent increases in markers of cholesterol absorption (e.g., campesterol, β-sitosterol). (See, e.g. Reihner E, Rudling M, Stalberg D, et al. Influence of pravastatin, a specific inhibitor of HMG-CoA reductase, on hepatic metabolism of cholesterol. N Engl J Med 1990; 323:224-8; Lamon-Fava S, Diffenderfer M R, Barrett P H, et al. Effects of different doses of atorvastatin on human apolipoprotein B-100, B-48, and A-I metabolism. J Lipid Res 2007; 48:1746-53; Ooi E M, Barrett P H, Chan D C, Nestel P J, Watts G F. Dose-dependent effect of rosuvastatin on apolipoprotein B-100 kinetics in the metabolic syndrome. Atherosclerosis 2008; 197:139-46; De Cuyper I, Wolthers B G, van Doormaal J J, Wijnandts P N. Determination of changes in serum lathosterol during treatment with simvastatin to evaluate the role of lathosterol as a parameter for whole body cholesterol synthesis. Clin Chim Acta 1993; 219:123-30.) The magnitude of change in these sterol markers has been reported to vary by statin dose, with lower doses having smaller effects. See Ibid. Differences between statins have also been observed. Atorvastatin was found to reduce serum lathosterol/cholesterol ratios more than simvastatin, while it increased plant sterol/cholesterol ratios more than simvastatin in patients with coronary heart disease. (See Miettinen T A, Gylling H, Lindbohm N, Miettinen T E, Rajaratnam R A, Relas H. Serum noncholesterol sterols during inhibition of cholesterol synthesis by statins. J Lab Clin Med 2003; 141:131-7.)

It has been previously reported that atorvastatin 80 mg/day and rosuvastatin 40 mg/day caused similar reduction in markers of cholesterol synthesis, but rosuvastatin increased markers of cholesterol absorption significantly less than atorvastatin. (See, van Himbergen T M, Matthan N R, Resteghini N A, et al. Comparison of the effects of maximal dose atorvastatin and rosuvastatin therapy on cholesterol synthesis and absorption markers. J Lipid Res 2009; 50:730-9.) That study also suggested that the combined effect of statins on cholesterol synthesis and absorption may influence treatment efficacy, since the greatest reduction in total cholesterol and LDL-C was seen in subjects with the largest reduction in lathosterol and no compensatory increase in campesterol while treatment efficacy was the lowest in subjects where the converse was true.

Ezetimibe is a selective cholesterol absorption inhibitor that blocks the transport of cholesterol and phytosterols across the intestinal wall and significantly reduces LDL-C levels by 15-20%. (See, e.g. Davidson M H, McGarry T, Bettis R, et al. Ezetimibe coadministered with simvastatin in patients with primary hypercholesterolemia. J Am Coll Cardiol 2002; 40:2125-34; Gagne C, Gaudet D, Bruckert E. Efficacy and safety of ezetimibe coadministered with atorvastatin or simvastatin in patients with homozygous familial hypercholesterolemia. Circulation 2002; 105:2469-75.) Ezetimibe decreases markers of cholesterol absorption but also produces a compensatory increase in markers of cholesterol synthesis. (See, Sudhop T, Lutjohann D, Kodal A, et al. Inhibition of intestinal cholesterol absorption by ezetimibe in humans. Circulation 2002; 106:1943-8.) Co-administration of ezetimibe with a statin has been shown to inhibit cholesterol absorption as well as synthesis and these complementary effects produce significantly greater reductions in LDL-C than either drug alone. (See, e.g. Gouni-Berthold I, Berthold H K, Gylling H, et al. Effects of ezetimibe and/or simvastatin on LDL receptor protein expression and on LDL receptor and HMG-CoA reductase gene expression: a randomized trial in healthy men. Atherosclerosis 2008; 198:198-207; Ballantyne C M, Houri J, Notarbartolo A, et al. Effect of ezetimibe coadministered with atorvastatin in 628 patients with primary hypercholesterolemia: a prospective, randomized, double-blind trial. Circulation 2003; 107:2409-15; Morrone D, Weintraub W S, Toth P P, Hanson M E, Lowe R S, Lin J, Shah A K, and Tershakovec A M. Lipid-altering efficacy of ezetimibe plus statin and statin monotherapy and identification of factors associated with treatment response: A pooled analysis of over 21,000 subjects from 27 clinical trials. Atherosclerosis, in press. 2012.)

The effects of ezetimibe added to different statins on cholesterol lowering and cholesterol homeostasis has not been well studied, especially not in a large head-to-head comparison study. The goals of this study were to compare the effects of adding ezetimibe 10 mg to different statins and doses on plasma lipid-lowering effects and non-cholesterol sterol levels. Subjects enrolled in the EASE study had LDL-C levels above National Cholesterol Education Program Adult Treatment Panel III (NCEP ATP III) recommended targets while on statin therapy, and were randomized to receive either placebo or ezetimibe in addition to their ongoing statin. (See Pearson T A, Denke M A, McBride P E, Battisti W P, Brady W E, Palmisano J. A community-based, randomized trial of ezetimibe added to statin therapy to attain NCEP ATP III goals for LDL cholesterol in hypercholesterolemic patients: the ezetimibe add-on to statin for effectiveness (EASE) trial. Mayo Clin Proc 2005; 80:587-95.) We used lipid and non-cholesterol sterol data from the ezetimibe arm of this study to test the hypothesis that ezetimibe, when added to statin therapy, would be most effective in LDL-C lowering in subjects on high potency statins and that these effects would be related to alterations in markers of cholesterol absorption (β-sitosterol, β-sitosterol/cholesterol) and synthesis (lathosterol, lathosterol/cholesterol).

### Methods

Subjects and Study Design

This study included subjects from the ezetimibe add-on to statin arm of the EASE study (http://clinicaltrials.gov identifier NCT00092586; Study Protocol 040). Details of the study design and outcomes have been published previously. (See Pearson T A, Denke M A, McBride P E, Battisti W P, Brady W E, Palmisano J. A community-based, randomized trial of ezetimibe added to statin therapy to attain NCEP ATP III goals for LDL cholesterol in hypercholesterolemic patients: the ezetimibe add-on to statin for effectiveness (EASE) trial. Mayo Clin Proc 2005; 80:587-95; Pearson T A, Denke M A, McBride P E, et al. Effectiveness of ezetimibe added to ongoing statin therapy in modifying lipid profiles and low-density lipoprotein cholesterol goal attainment in patients of different races and ethnicities: a substudy of the Ezetimibe add-on to statin for effectiveness trial. Mayo Clin Proc 2006; 81:1177-85.)

Briefly, the EASE study was a multicenter, randomized, double-blind, placebo-controlled, 6-week parallel-group study. Participants with hypercholesterolemia were recruited from community based practices across the United States. Inclusion criteria were: 1) age ≧18 years, 2) on a stable, approved dose of any statin, 3) following a cholesterol-lowering diet for ≧6 weeks before study entry, and 4) LDL-C levels above risk-based NCEP ATP III targets. Subjects receiving lipid-altering agents other than statins during the 6 weeks before screening were excluded. Patients were randomized to receive ezetimibe 10 mg/day or placebo plus their current statin therapy and dose for 6 weeks. Statin type and dose were maintained throughout the study. The research protocol was approved by the investigational review boards at each site, and all participants provided written informed consent prior to study start.

For this current post hoc analysis, we assessed subjects who were randomized into the ezetimibe 10 mg plus statin arm of the EASE study (n=1940). Of the 1124 subjects with samples available for measurement, only those who had complete sterol and lipid data at baseline and at the end of the 6-week study were included in this analysis (n=874). The mean age and other baseline characteristics of those included and excluded from this analysis were similar. Comparison of subjects was based on statin type or potency (low, medium, high) subgroups. The low potency statin group (predicted LDL-C reduction of ˜20-30%) included subjects receiving simvastatin ≦10 mg/day, lovastatin ≦20 mg/day, pravastatin ≦20 mg/day, and fluvastatin ≦40 mg/day. The medium potency statin group (predicted LDL-C reduction of ˜31-45%) included subjects receiving simvastatin >10 to ≦40 mg/day, atorvastatin ≦20 mg/day, lovastatin >20 to 80 mg/day, pravastatin >20 to 80 mg/day, and fluvastatin >40 to 80 mg/day. The high potency statin group (predicted LDL-C reduction of ˜46-55%) included subjects receiving simvastatin >40 to 80 mg/day, and atorvastatin >20 to 80 mg/day.

Measurement of Lipoproteins and Non-Cholesterol Sterols

Plasma total cholesterol (total C), high-density lipoprotein cholesterol (HDL-C), and triglycerides (TG) were analyzed using standardized methods at the central laboratory of the trial (PPD Global Central Labs, Highland Heights, Kentucky, USA). LDL-C was calculated using the Friedewald formula. (See Friedewald W T, Levy R I, Fredrickson D S. Estimation of the concentration of low-density lipoprotein cholesterol in plasma, without use of the preparative ultracentrifuge. Clin Chem 1972; 18:499-502.) Non-HDL-C was calculated by subtracting HDL-C from total C. Apolipoprotein (Apo) A-I, Apo B, and high-sensitivity C-reactive protein (hs-CRP) were measured by automated immunoassays at the central laboratory. Within and between coefficients for all assays were <10%. Plasma lathosterol and β-sitosterol were quantified by gas chromatography mass spectrometry after lipid extraction as previously described in Sudhop T, Lutjohann D, Kodal A, et al. Inhibition of intestinal cholesterol absorption by ezetimibe in humans. Circulation 2002; 106:1943-8. Since these plasma sterols are mainly carried in the LDL fraction (see, e.g. Miettinen T A, Tilvis R S, Kesaniemi Y A. Serum plant sterols and cholesterol precursors reflect cholesterol absorption and synthesis in volunteers of a randomly selected male population. Am J Epidemiol 1990; 131:20-31), it is common practice to adjust them for total plasma cholesterol by expressing them as a ratio to cholesterol, or to express them as a ratio representing both absorption and synthesis (i.e. sitosterol/lathosterol). Plasma sterols were therefore expressed either in absolute terms, as a ratio to cholesterol, or as a ratio of β-sitosterol to lathosterol.

Statistical Analysis

All continuous variables were expressed as means±standard deviation (SD) for normally distributed data, or medians±robust SD if non-normally distributed. Subjects receiving lovastatin and fluvastatin were grouped together as “other statins” when evaluating statin types due to the small number of subjects. For samples with sterol levels below the limit of detection (0.5 μg/ml) either at the time of randomization or at study end, a value of 0.25 μg/ml was assigned to prevent any bias of excluding subjects with very low sterol levels. All subjects were receiving statin therapy at study entry and baseline values represent levels while on treatment. Baseline cholesterol synthesis and absorption markers among the different statins were compared using an ANOVA model with terms for statin type and statin dose within statin type. Changes from baseline in plasma lipids, apolipoproteins and hs-CRP after ezetimibe treatment were assessed using an ANOVA model with terms for statin type or statin potency. Data were presented as mean and 95% confidence interval (CI) or median and 95% CI for non-normally distributed data. Since baseline LDL-C levels can influence the lipid-lowering effect of hypolipidemic agents, data were also calculated as percent changes from baseline. Lipid data were also calculated by adjustment for lipid values from the placebo arm of the original EASE study. ANOVA with term for statin type or statin potency were used to compare changes from baseline of plasma lipid values among groups as well as changes in lathosterol and β-sitosterol. Correlation between changes from baseline of these sterols with changes of plasma lipids values were assessed using Pearson's correlation. Multivariate analysis was performed to assess factors associated with changes in plasma lathosterol and β-sitosterol levels with an add-on ezetimibe treatment.

### Results

Effect of Statin Potency, Type, and Dose on Baseline Lipids and Sterols

Baseline characteristics, lipid values and non-cholesterol sterol levels for the overall population are presented in Tables 1A-B.

TABLE 1A
Baseline Characteristics, lipids, and sterols. A. Overall Subgroups by Statin Potency.

| | | Statin Potency | | |
|---|---|---|---|---|
| | | | Medium | |
| Characteristic | Overall n = 874 | Low (N = 133) | (N = 582) | High (N = 159) |
| Mean age (yr) | 61.3 ± 11.2 | 62.0 ± 11.2 | 61.4 ± 11.2 | 60.5 ± 11.0 |
| Male (%) | 473 (54.1) | 68 (51.1) | 312 (53.6) | 93 (58.5) |
| Race: Caucasian, n | 722 (82.6) | 97 (72.9) | 485 (83.3) | 140 (88.1) |
| (%) | | | | |
| Race: African | 67 (7.7) | 10 (7.5) | 45 (7.7) | 12 (7.5) |
| American, n (%) | | | | |
| Race: Others, n (%) | 85 (9.7) | 26 (19.5) | 52 (8.9) | 7 (4.4) |
| DM, n (%) | 361 (41.3) | 61 (45.9) | 253 (43.5) | 47 (29.6) |
| Body mass index | 30.8 ± 6.6 | 30.1 ± 7.5 | 31.0 ± 6.5 | 30.7 ± 6.1 |
| (kg/m²) | | | | |
| Total C (mg/dl)* | 210.7 ± 34.2 | 215.8 ± 35.2 | 208.7 ± 32.4 | 213.6 ± 39.1 |
| LDL-C (mg/dl)** | 129.5 ± 29.0 | 135.1 ± 30.1 | 127.0 ± 26.4 | 134.1 ± 35.2 |
| Triglyceride | 151.0 ± 83.7 | 152.0 ± 75.3 | 151.0 ± 81.9 | 150.0 ± 91.2 |
| (mg/dl)^{a} | | | | |
| HDL-C (mg/dl) | 48.1 ± 11.3 | 47.9 ± 11.9 | 48.6 ± 11.3 | 46.5 ± 10.5 |
| Total C/HDL-C | 4.56 ± 1.112 | 4.74 ± 1.29 | 4.46 ± 1.06 | 4.76 ± 1.15 |
| ratio** | | | | |
| Non-HDL-C | 162.5 ± 33.1 | 167.8 ± 35.8 | 160.1 ± 31.0 | 167.1 ± 37.0 |
| (mg/dl)** | | | | |
| Apo B (mg/dl)** | 129.2 ± 24.8 | 132.1 ± 27.1 | 127.3 ± 23.6 | 133.8 ± 26.7 |
| Apo A1 (mg/dl)* | 158.1 ± 26.9 | 157.8 ± 7.2 | 159.7 ± 27.1 | 152.4 ± 25.2 |
| hs-CRP (mg/L)^{a} | 2.50 ± 3.72 | 2.80 ± 3.91 | 2.50 ± 3.53 | 2.40 ± 4.37 |
| Lathosterol | 2.55 ± 1.70 | 3.17 ± 1.70 | 2.58 ± 1.70 | 1.93 ± 1.63 |
| (μmol/L)*** | | | | |
| B-sitosterol | 4.86 ± 2.62 | 4.51 ± 2.03 | 4.58 ± 2.46 | 6.21 ± 3.14 |
| (μmol/L)*** | | | | |
| Lathosterol/Total C | 0.46 ± 0.27 | 0.56 ± 0.27 | 0.47 ± 0.27 | 0.34 ± 0.24 |
| (μmol/mmol)*** | | | | |
| B-sitosterol/Total | 0.91 ± 0.50 | 0.83 ± .39 | 0.86 ± 0.47 | 1.15 ± 0.60 |
| C (μmol/mmol)*** | | | | |
| B-sitosterol/ | 3.16 ± 3.53 | 2.03 ± 2.22 | 2.77 ± 3.08 | 5.50 ± 4.78 |
| Lathosterol | | | | |

*ρ < 0.015;
**ρ < 1.01;
***ρ < 1.001 indicate significant differences between statin potency groups by ANOVA.
Apo, apolipoprotein;
HDL-C, high density lipoprotein cholesterol;
hs-CRP, high sensitivity C-reactive protein;
LDL-C, low density lipoprotein cholesterol;
Total C, total cholesterol.
Values are expressed as mean ± standard deviation (SD)
^{a}Median ± robust SD

TABLE 1B
Baseline Characteristics, lipids, and sterols. A. Overall Subgroups by Statin Type

| | Atorvastatin | Simvastatin | Pravastatin | |
|---|---|---|---|---|
| Characteristic | (n = 345) | (n = 233) | (n = 209) | Other Statins^{a} |
| Mean age (yr) | 60.0 ± 11.7 | 61.6 ± 10.3 | 61.6 ± 11.4 | 63.2 ± 10.2 |
| Male (%) | 182 (52.8%) | 143 (61.4%) | 104 (49.8%) | 44 (50.6%) |
| Race: Caucasian, n | 286 (82.9) | 195 (83.7) | 167 (79.9) | 74 (85.1) |
| (%) | | | | |
| Race: African | 29 (8.4) | 19 (8.2) | 14 (6.7) | 5 (5.7) |
| American, n (%) | | | | |
| Race: Others, n (%) | 30 (8.7) | 19 (8.2) | 28 (13.4) | 8 (9.2) |
| DM, n (%) | 136 (39.4) | 98 (42.1) | 88 (42.1) | 39 (44.8) |
| Body mass index | 31.1 ± 6.5 | 30.8 ± 6.5 | 30.2 ± 5.9 | 30.9 ± 8.8 |
| (kg/m²) | | | | |
| Total C (mg/dl)* | 209.7 ± 35.4 | 205.5 ± 31.4 | 215.5 ± 34.9 | 216.4 ± 33.2 |
| LDL-C (mg/dl)** | 130.0 ± 30.9 | 124.3 ± 26.1 | 133.1 ± 28.3 | 133.4 ± 28.1 |
| Triglyceride | 149.0 ± 85.6 | 149.0 ± 88.4 | 155.0 ± 80.0 | 157.0 ± 69.8 |
| (mg/dl)^{a} | | | | |
| HDL-C (mg/dl) | 47.8 ± 10.7 | 48.2 ± 11.8 | 48.6 ± 12.0 | 48.4 ± 10.3 |
| Total C/HDL-C | 4.55 ± 1.05 | 4.46 ± 1.10 | 4.65 ± 1.25 | 4.64 ± 1.09 |
| ratio** | | | | |
| Non-HDL-C | 162.0 ± 33.6 | 157.4 ± 30.2 | 166.9 ± 34.5 | 168.0 ± 33.1 |
| (mg/dl)** | | | | |
| Apo B (mg/dl)** | 129.2 ± 25.4 | 125.8 ± 23.2 | 131.3 ± 26.0 | 133.4 ± 23.0 |
| Apo A1 (mg/dl)* | 156.3 ± 26.1 | 157.7 ± 26.3 | 160.5 ± 28.2 | 160.0 ± 28.0 |
| hs-CRP (mg/L)^{b} | 2.40 ± 3.53 | 2.50 ± 3.81 | 2.70 ± 4.09 | 2.50 ± 3.53 |
| Lathosterol | 2.20 ± 1.70 | 2.47 ± 1.68 | 3.08 ± 1.69 | 2.90 ± 1.42 |
| (μmol/L)*** | | | | |
| B-sitosterol | 5.40 ± 2.93 | 4.33 ± 2.17 | 4.72 ± 2.51 | 4.48 ± 2.25 |
| (μmol/L)*** | | | | |
| Lathosterol/Total C | 0.39 ± 0.26 | 0.46 ± 0.28 | 0.55 ± 0.26 | 0.52 ± 0.24 |
| (μmol/mmol)*** | | | | |
| B-sitosterol/Total | 1.01 ± 0.57 | 0.82 ± 0.40 | 0.86 ± 0.48 | 0.81 ± 0.42 |
| C (μmol/mmol)*** | | | | |
| B-sitosterol/ | 4.22 ± 4.39 | 2.86 ± 296 | 2.11 ± 2.10 | 2.23 ± 2.62 |
| Lathosterol | | | | |

*ρ < 0.015;
**ρ < 1.01;
***ρ < 1.001 indicate significant differences between statin potency groups by ANOVA.
Apo, apolipoprotein;
HDL-C, high density lipoprotein cholesterol;
hs-CRP, high sensitivity C-reactive protein;
LDL-C, low density lipoprotein cholesterol;
Total C, total cholesterol.
Values are expressed as mean ± standard deviation (SD)
^{a}Other statins include lovastatin and fluvastatin
^{b}Median ± robust SD

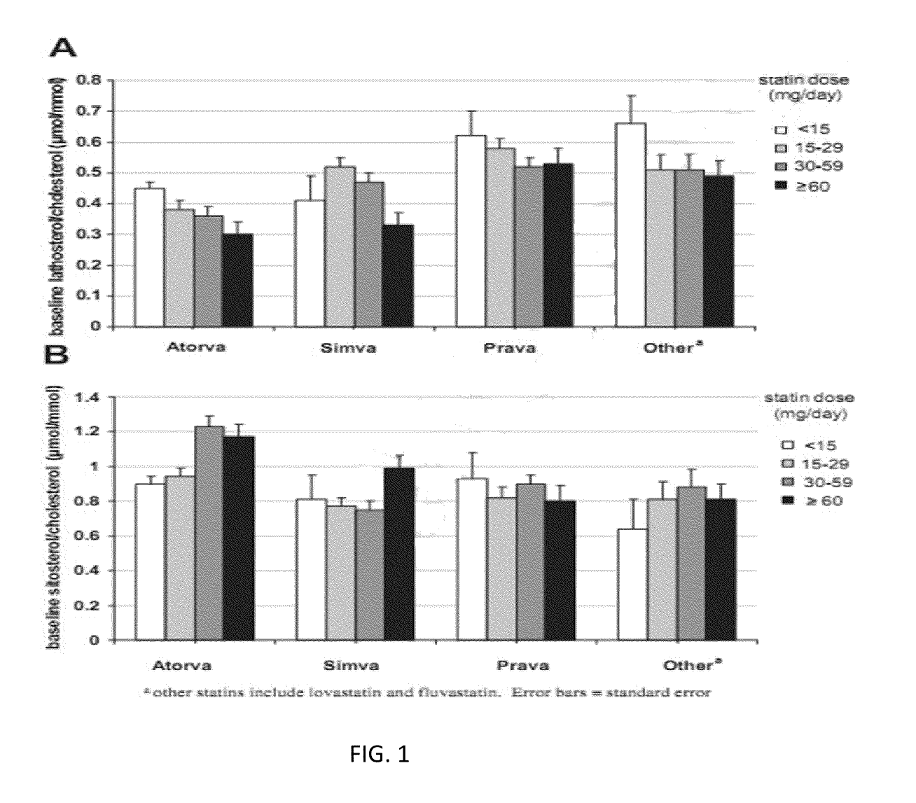

The mean age was 61.3 years old, 54.1% were male, and the majority of subjects were Caucasian (82.6%, 7.7% were African American and 9.7% were other ethnic groups). Mean lipid levels were 210.7 mg/dl for total C, 129.5 mg/dl for LDL-C, and 48.1 mg/dL for HDL-C, while the median TG level was 151.0 mg/dl. Of the 874 subjects studied, 133 (15.2%) were receiving low potency statins, 582 (66.6%) were receiving medium potency statins, and 159 (18.2%) were receiving high potency statins (Table 1A). Subjects in the low and high potency statin groups had similar baseline lipid values, while subjects in the medium potency statin group had lower baseline total C, LDL-C, non-HDL-C, total C/HDL-C ratio, and Apo B levels based on post hoc Tukey analysis. Subjects receiving high potency statins also had lower Apo A-1 levels than those in the medium potency statin group. The distribution of subjects by statin type were 345 (39.5%) for atorvastatin, 233 (26.7%) for simvastatin, 209 (23.9%) for pravastatin, and 87 (9.9%) for lovastatin or fluvastatin (Table 1B). Subjects on simvastatin had significantly lower baseline total C, LDL-C, non-HDL-C, and Apo B levels than those receiving other statin types. Significant differences in baseline cholesterol synthesis and absorption marker levels were seen when evaluating statin potency, type, and dose. Subjects in the high potency statin group had significantly lower baseline lathosterol and lathosterol/cholesterol levels (p<0.001) and significantly higher β-sitosterol, β-sitosterol/cholesterol, and sitosterol/lathosterol levels (p<0.001) than subjects in the low potency statin group (Table 1A). Mean values for high vs. medium vs. low potency statin groups were 0.34 vs. 0.47 vs. 0.56 μmol/mmol for baseline lathosterol/cholesterol levels, 1.15 vs. 0.86 vs. 0.83 μmol/mmol for baseline β-sitosterol/cholesterol levels, and 5.50 vs. 2.77 vs. 2.03 for baseline β-sitosterol/lathosterol, respectively. In addition, mean lathosterol/cholesterol ratios were significantly lower (p <0.0001) while mean β-sitosterol/cholesterol and β-sitosterol/lathosterol ratios were significantly higher (p<0.0001) in those who used atorvastatin than those who used other statins (Table 1B). In an ANOVA model, statin type and statin dose used within statin type were significantly associated with the ratios of lathosterol/cholesterol and β-sitosterol/cholesterol (p<0.001). These findings were also confirmed when these markers were assessed in absolute terms. Baseline lathosterol/cholesterol and β-sitosterol/cholesterol ratios classified by the different statins and their dosage further demonstrate that statin type and dose are significantly associated with levels of cholesterol synthesis and absorption markers (FIG. 1, Panels A and B).

Effects of Ezetimibe Add-on Therapy: Plasma Lipids, Apolipoproteins, and hs-CRP

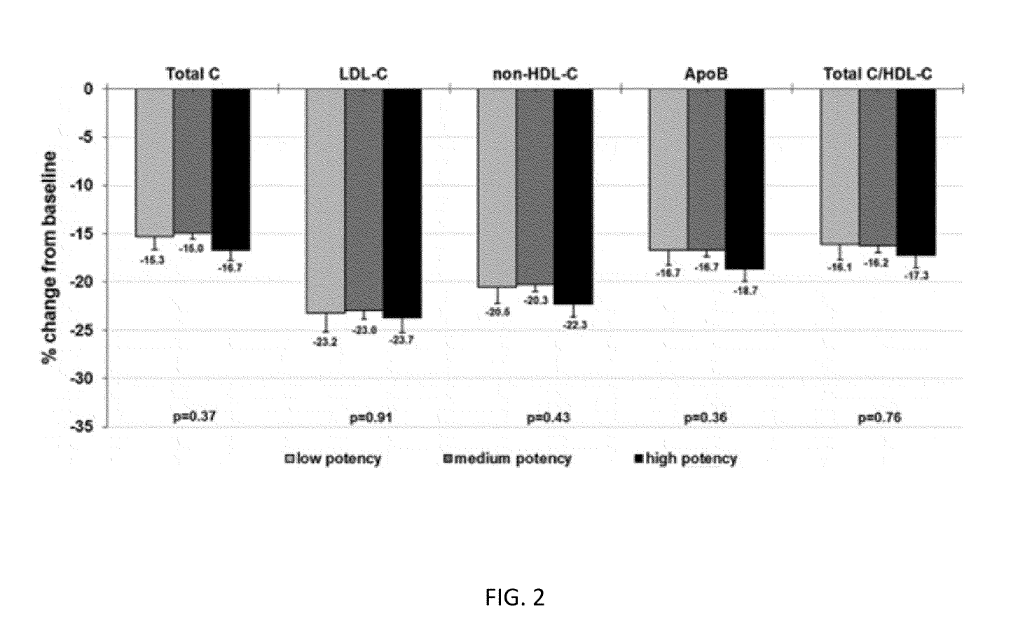

Evaluation of data from the ezetimibe add-on to ongoing statin treatment arm of the EASE study showed significant reductions in total C, LDL-C, TG, non-HDL-C, total C/HDL-C, and Apo B from baseline across all statin potency groups. The high potency statin group, however, showed significantly greater reductions in total C, LDL-C, non HDL-C, Apo B, and total C/HDL-C than observed for the other groups. Since baseline LDL-C levels are known to affect the LDL-C lowering response of hypolipidemic agents, percent reduction of these lipid values were also evaluated and found to be significantly greater in the high potency group. The mean percent LDL-C reduction in the high, medium, and low potency statin groups were 29.1%, 25.0% and 22.7%, respectively (p=0.002). When results were adjusted for lipid values from the placebo arm of the EASE study, the high potency statin group had the greatest reductions in total C, LDL-C, non-HDL-C, Apo B, and total C/HDL-C; however, these differences were no longer statistically significant from the other groups (FIG. 2). This result may be due in part to modest reductions from baseline in total C, LDL-C, non-HDL-C, Apo B and total C/HDL-C that were observed with placebo treatment, with the greatest effect seen in the high potency statin group.

The addition of ezetimibe to all statin types resulted in significant reductions from baseline in total C, LDL-C, non-HDL-C, Apo B, and total C/HDL-C with no between-type differences (Supplementary Table 1). The percent LDL-C reductions from baseline for ezetimibe combinations with atorvastatin, pravastatin, simvastatin, and lovastatin or fluvastatin were 26.4%, 23.1%, 26.4%, and 24.1% respectively, p=0.08. Response to ezetimibe add-on therapy was also evaluated in subgroups of subjects with statin treated baseline absorption marker levels above the median (potentially higher absorbers) or ≦the median (potentially lower absorbers) in order to determine if absorption marker status was associated with treatment efficacy. No significant between group differences were seen for any of the baseline absorption marker groups (sitosterol, sitosterol/cholesterol, sitosterol/lathosterol) when evaluating absolute change from baseline in total cholesterol, LDL-C, triglycerides, non-HDL-C, ApoB and total cholesterol/HDL-C. Assessment of percent change from baseline showed small but significantly greater reductions in LDL-C (−26.7% vs. −24.1%), non-HDL-C (−24.2% vs. −21.6%) and total C/HDL-C (−19.0% vs. −17.0%) for subjects with baseline sitosterol/lathosterol levels above the median, while no significant differences were seen when evaluating subgroups based on baseline sitosterol or sitosterol/cholesterol marker levels.

Effects of Ezetimibe Add-on Therapy: Markers of Cholesterol Absorption and Synthesis

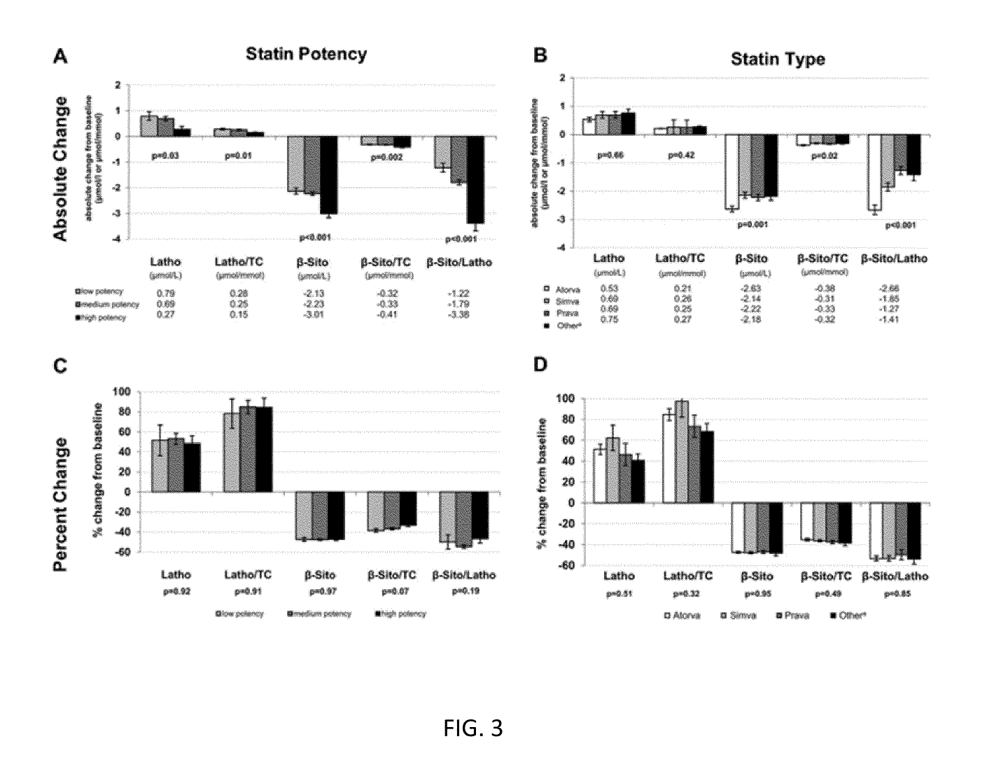

Add-on ezetimibe therapy resulted in significant increases in cholesterol synthesis markers and significant reductions in cholesterol absorption markers from baseline for each statin type and statin potency level. Mean lathosterol levels in the overall study population increased by 52% (95% CI; 42.9, 61.0) and the mean lathosterol/cholesterol ratio increased by 83.8% (95% CI; 73.3, 94.2). Mean β-sitosterol levels decreased by 47.4% (95% CI; −48.7, −46.2) and the mean β-sitosterol/cholesterol ratio decreased by 36.3% (95% CI; −37.7, −34.9). Overall percent change in the mean β-sitosterol/lathosterol ratio was −52.4% (95% CI; −55.8, −49.0). All sterol changes were highly significant (p<0.001). Addition of ezetimibe to high potency statins produced significantly lower increases in cholesterol synthesis markers than seen with medium and low potency statins (FIG. 3, Panel A). In addition, combination therapy with high potency statins resulted in significantly greater reductions in cholesterol absorption markers and β-sitosterol/lathosterol than medium and low potency statins. When changes in plasma non-cholesterol levels from baseline classified by statin type were compared, there were no significant differences between lathosterol and lathosterol/cholesterol among the four groups (FIG. 3, Panel B). Adding ezetimibe to atorvastatin decreased β-sitosterol, β-sitosterol/cholesterol, and β-sitosterol/lathosterol levels more than when added to the other statins, but there were no significant differences among other stain types. Evaluation of percent change from baseline in plasma non-cholesterol levels showed no significant difference between statin potencies or statin types (FIG. 3, Panels C and D).

Correlation Between Lipid-Lowering Efficacy and Changes in Cholesterol Synthesis and Absorption Markers

Tables 2A and 2B shows the correlations between changes in plasma lipids and sterols overall and classified by statin potency groups after 6 weeks of ezetimibe-add on therapy.

### Tables 2A and 2B

Correlation between changes of cholesterol synthesis and absorption markers with changes of plasma lipids and apo B levels after 6 weeks of ezetimibe add-on therapy.

TABLE 2A

| | | | Medium | High |
|---|---|---|---|---|
| Parameter | All | Low potency | potency | potency |
| | Changes in β-sitosterol | | | |
| Changes in Total C | 0.260** | 0.364** | 0.229** | 0.217* |
| Changes in LDL-C | 0.245** | 0.346** | 0.207** | 0.208* |
| Changes in Triglycerides | 0.073* | 0.043 | 0.057 | 0.144 |
| Changes in HDL-C | 0.061 | 0.003 | 0.103* | −0.033 |
| Changes in Total C/HDL-C | 0.170** | 0.207* | 0.133* | 0.177* |
| Changes in Non-HDL-C | 0.250** | 0.359** | 0.210** | 0.222* |
| Changes in Apo B | 0.218** | 0.258* | 0.189** | 0.191* |
| | Changes in β-sitosterol/cholesterol | | | |
| Changes in Total C | −0.079* | 0.077 | −0.071 | −0.229* |
| Changes in LDL-C | −0.064 | 0.085 | −0.052 | −0.229* |
| Changes in Triglycerides | −0.043 | −0.026 | −0.067 | 0.016 |
| Changes in HDL-C | 0.029 | 0.018 | 0.059 | −0.063 |
| Changes in Total C/HDL-C | −0.061 | 0.001 | −0.075 | −0.107 |
| Changes in Non-HDL-C | −0.086* | 0.072 | −0.086 | −0.217* |
| Changes in Apo B | −0.093* | 0.002 | −0.085* | −0.231* |

Values are expressed as correlation coefficients.
*p < 0.05:
**p < 0.001.

TABLE 2B

| | | | Medium | High |
|---|---|---|---|---|
| Parameter | All | Low potency | potency | potency |
| | Changes in lathosterol | | | |
| Changes in Total C | 0.339** | 0.203* | 0.291** | 0.646** |
| Changes in LDL-C | 0.288** | 0.146 | 0.240** | 0.601** |
| Changes in Triglycerides | 0.164** | 0.149 | 0.161** | 0.220* |
| Changes in HDL-C | 0.022 | −0.068 | 0.039 | 0.016 |
| Changes in Total C/HDL-C | 0.241** | 0.204* | 0.186** | 0.485** |
| Changes in Non-HDL-C | 0.338** | 0.216* | 0.288** | 0.641** |
| Changes in Apo B | 0.311** | 0.131 | 0.274** | 0.610** |
| | Changes in lathosterol/cholesterol | | | |
| Changes in Total C | 0.103* | −0.066 | 0.076 | 0.382** |
| Changes in LDL-C | 0.086* | −0.099 | 0.065 | 0.354** |
| Changes in Triglycerides | 0.068* | 0.126 | 0.058 | 0.111 |
| Changes in HDL-C | −0.024 | −0.139 | −0.012 | −0.005 |
| Changes in Total C/HDL-C | 0.076* | 0.069 | 0.033 | 0.290** |
| Changes in Non-HDL-C | 0.109* | −0.036 | 0.080 | 0.381** |
| Changes in Apo B | 0.106** | −0.090 | 0.088* | 0.374** |

Values are expressed as correlation coefficients.
*p < 0.05:
**p < 0 0.001.

Changes in lathosterol and β-sitosterol levels were significantly correlated with changes in total C, LDL-C, non-HDL-C, total C/HDL-C and Apo B for the overall population (all p<0.001). Correlations with changes in TG were significant but considerably weaker. Correlations of changes in lathosterol with these plasma lipid values were strongest in the high potency statin group while correlations of changes in β-sitosterol with plasma lipid values were strongest in the low potency statin group. Correlations of changes in lathosterol/cholesterol were also significantly associated with changes in total C, LDL-C, non-HDL-C, total C/HDL-C and Apo B overall, but were weaker than the absolute values and remained significant only for high and medium (Apo B only) potency statin groups (Tables 2A and 2B) and ezetimibe-atorvastatin group. Changes in β-sitosterol/cholesterol were negatively and weakly correlated with changes in total C, non-HDL-C and Apo B overall. Again, only the high and medium (Apo B only) potency statin and ezetimibe-atorvastatin groups were significant.

Factors Related to Changes in Cholesterol Synthesis and Absorption Markers

Multivariate analysis was used to assess factors associated with changes in lathosterol and β-sitosterol after 6 weeks of ezetimibe add-on therapy (Table 3).

TABLE 3
Multivariate analysis for variables associated with changes in lathosterol
and β-sitosterol after 6 weeks of ezetimibe add-on therapy.

| | Change in lathosterol | | Change in β-sitosterol | |
|---|---|---|---|---|
| Factor | Beta | P value | Beta | P value |
| Baseline total | 0.009 | 0.0350 | 0.002 | 0.1936 |
| Baseline LDL-C | 0.004 | 0.4538 | 0.005 | 0.0210 |
| Changes in | 0.026 | <0.0001 | 0.012 | <0.0001 |
| Changes in LDL-C | 0.001 | 0.8297 | 0.005 | 0.1015 |
| Baseline lathosterol | −0.409 | <0.0001 | | |
| Baseline β-sitosterol | | | −0.540 | <0.0001 |
| Statin type | | 0.5976 | | 0.7147 |
| Statin potency | | | | |
| High-potency | −0.491 | 0.0065 | 0.201 | 0.0215 |
| Medium-potency | 0.000 | | 0.000 | |
| Low-potency | 0.230 | | 0.004 | |

Factors significantly associated with changes in lathosterol were baseline total cholesterol, changes in total cholesterol, baseline lathosterol, and statin potency. Factors significantly associated with changes in β-sitosterol were baseline LDL-C, changes in total cholesterol, baseline β-sitosterol, and statin potency. For changes in lathosterol and β-sitosterol, the baseline values of lathosterol and β-sitosterol, respectively, were the strongest predictors during ezetimibe add-on treatment. Changes in LDL-C and stain type were not significantly associated with any sterol change.

### Discussion

This post hoc analysis of the EASE study evaluated the effects of ezetimibe on lipid and sterol markers when added to ongoing statin treatment. To our knowledge, this is the first head-to-head comparison evaluating the effect of ezetimibe add-on to low, medium and high potency statins on changes in cholesterol synthesis and absorption markers and LDL-C. Although it is the second study comparing the addition of ezetimibe to a variety of statins on markers of cholesterol synthesis and absorption, the previous study evaluated the effects of statin, ezetimibe, or both in subjects with primary hypercholesterolemia (See, Assmann G, Kannenberg F, Ramey D R, Musliner T A, Gutkin S W, Veltri E P. Effects of ezetimibe, simvastatin, atorvastatin, and ezetimibe-statin therapies on non-cholesterol sterols in patients with primary hypercholesterolemia. Curr Med Res Opin 2008; 24:249-59) while the current study assessed the effects of ezetimibe add-on therapy in subjects already on statins and above NCEP ATP III recommended LDL-C targets.

As expected, our study found that addition of ezetimibe to ongoing atorvastatin, simvastatin, pravastatin, and lovastatin or fluvastatin resulted in significant LDL-C lowering from treated baseline and no significant differences between statins were observed. A pooled analysis from 4 different statin trials in patients with primary hypercholesterolemia also found that ezetimibe 10 mg/day co-administered with lovastatin, simvastatin, pravastatin, or atorvastatin resulted in similar percentage LDL-C reduction from baseline across statin types and statin doses. We hypothesized, however, that the addition of ezetimibe to high potency statins would result in greater LDL-C reductions than seen with medium or low potency statins, and that these effects should be related to alterations in markers of cholesterol synthesis and absorption. Statins inhibit cholesterol synthesis but effects on lowering LDL-C may be moderated by a compensatory up-regulation of cholesterol absorption, with higher potency statins having a greater effect. Ezetimibe add-on therapy might therefore be expected to have the greatest LDL-C lowering effect in subjects with higher levels of cholesterol absorption induced by high potency statins.

In the current study, subjects receiving high potency statins had significantly higher levels of cholesterol absorption markers (β-sitosterol and β-sitosterol/cholesterol) and lower levels of cholesterol synthesis markers (lathosterol, lathosterol/cholesterol) compared with lower potency statin groups. These results are consistent with studies comparing different doses of atorvastatin and rosuvastatin which found that higher doses of statin resulted in greater reductions in cholesterol synthesis marker levels and greater increases in cholesterol absorption marker levels than lower doses of the same statin. In addition, the Scandinavian Simvastatin Survival Study (4S) also showed that the higher the dose, the longer the treatment period, and the higher the baseline absorption sterol ratios, the higher was the relative increase of absorption sterol ratios in subjects treated with simvastatin. (See Miettinen T A, Strandberg T E, Gylling H. Noncholesterol sterols and cholesterol lowering by long-term simvastatin treatment in coronary patients: relation to basal serum cholestanol. Arterioscler Thromb Vasc Biol 2000; 20:1340-6).

We found that addition of ezetimibe to high potency statins resulted in greater reductions in cholesterol absorption markers and smaller increases in cholesterol synthesis markers than observed with medium and low potency statins. Greater reductions in LDL-C levels were also observed for this high potency statin group compared with medium and low potency groups. However, these results did not reach statistical significance when adjusted for LDL-C changes observed in the placebo arm of the study. The differential effect of placebo on change from baseline LDL-C in the low (+0.1%), medium (−2.0%), and high (−3.3%) potency statin groups may have contributed to the lack of significance after adjustment. Substantial variability in response to both statin and ezetimibe therapy is well known, and future assessment of individual changes rather than group comparisons may provide additional insight into the effects of ezetimibe when administered with statins of differing potencies.

Controversy exists whether baseline non-cholesterol sterol levels predict LDL-C lowering response to statin and/or ezetimibe treatment. Miettinen and colleagues have proposed that subjects with high cholesterol absorption and low synthesis respond poorly to statins and may need combination therapy to lower serum cholesterol more effectively and prevent an increase in the levels of plant sterols. Some studies do not support the benefit of using baseline cholesterol absorption marker levels in predicting response to statin and ezetimibe/simvastatin treatment. However, two recent studies suggest that statins were most effective in subjects who did not upregulate cholesterol absorption during statin therapy.

Subjects enrolled in the EASE study were above NCEP ATP III recommended LDL-C levels while on statins, and therefore may represent less responsive patients with increased levels of cholesterol absorption, but other factors cannot be ruled out. Within this population, comparison of subjects with baseline cholesterol absorption marker levels either above or ≦the median found no significant differences in the LDL-C lowering response to ezetimibe add-on therapy when evaluating absolute change from statin treated baseline, and minimally significant differences when assessing percent change (only seen for sitosterol/lathosterol subgroups). Our post hoc evaluation of EASE was not designed to evaluate the ability of baseline non-cholesterol levels to predict the therapeutic efficacy of statins or ezetimibe because we did not measure baseline values of cholesterol synthesis and absorption markers before statin therapy was initiated, and further studies are needed to address this issue.

We did find that statin type affected levels of cholesterol synthesis and absorption markers. Subjects who were on atorvastatin had lower lathosterol/cholesterol and higher β-sitosterol/cholesterol levels than subjects who were on other statins. In concurrence with our findings, previous studies have shown that atorvastatin reduced precursor sterols more than simvastatin (e.g. −50% vs. −42% when comparing sterol/cholesterol ratios), and plant sterols increased more with atorvastatin than with simvastatin (e.g. 82% vs. 39% when comparing sterol/cholesterol ratios). We also found that addition of ezetimibe to atorvastatin produced significantly greater reduction of β-sitosterol, β-sitosterol/cholesterol, and β-sitosterol/lathosterol levels than when added to other statins, while increases in markers of cholesterol synthesis were similar across all statins. Despite these differences, addition of ezetimibe to any statin lowered LDL-C by 23-26% and no significant differences between statins were observed. Few other studies have evaluated the influence of statin type on cholesterol synthesis and absorption marker levels during combination therapy with ezetimibe, and none have assessed concomitant changes in LDL-C. In one post hoc evaluation of subjects with primary hypercholesterolemia, ezetimibe plus atorvastatin reduced lathosterol by 62.4% and β-sitosterol by 49.4% while co-administration of ezetimibe and simvastatin reduced lathosterol by 47.6% and β-sitosterol by 52.1%, supporting the current finding that different statins may differentially influence markers of cholesterol synthesis and absorption.

Common factors that were significantly associated with changes in lathosterol and β-sitosterol included baseline values of those sterols, changes in total cholesterol, and statin potency. Baseline sterol levels were the strongest predictors of sterol changes during an ezetimibe add-on treatment, indicating that baseline lathosterol may be related to changes in cholesterol synthesis and baseline β-sitosterol to reduction of cholesterol absorption. In addition, changes in sterol levels significantly correlated with changes in LDL-C following ezetimibe add-on therapy. A previous study also reported correlations of baseline non-cholesterol sterol levels with changes in these sterols and sterol changes with those of LDL-C during treatment with ezetimibe and simvastatin. It should be noted that the baseline sterol levels in this study were obtained during statin treatment prior to the addition of ezetimibe, while the baseline values in other studies were obtained before initiation of any therapy.

While this is one of the few studies to assess the effects of ezetimibe add-on therapy on both lipids and sterol markers, certain limitations were present. This study was a post hoc analysis of the EASE trial. Since subjects enrolled in this study had LDL-C levels above NCEP ATP III recommended targets while on statin therapy, results may not be generalizable to other patient populations and may potentially include some bias. Non-cholesterol sterol data was not available for the entire study cohort which could potentially bias the results, however no significant differences in baseline characteristics were seen between those included and excluded from the analysis. In addition, non-cholesterol sterols were not measured in the placebo arm; therefore we were unable to determine placebo-adjusted values for non-cholesterol sterols after the addition of ezetimibe. Further prospective randomized studies are needed to clarify this issue. The treatment duration of ezetimibe in this study was 6 weeks, and thus longer-term changes in these sterol markers were not assessed. However, a previous study has reported similar changes of cholesterol synthesis and absorption markers levels between short-term therapy and 2-year treatment in subjects with familial hypercholesterolemia. (See, Jakulj L, Vissers M N, Groen A K, et al. Baseline cholesterol absorption and the response to ezetimibe/simvastatin therapy: a post-hoc analysis of the ENHANCE trial. J Lipid Res 2010; 51:755-62.) Finally, we did not have baseline values for cholesterol synthesis and absorption markers prior to the initiation of statin therapy and therefore were not able to determine if baseline levels predict LDL-C lowering response to overall treatment. We did, however, have the values of the sterol markers during on-going statin therapy thus providing responses of the subjects to ezetimibe treatment.

### CONCLUSIONS

Results from this study indicate that statin potency and type can significantly affect cholesterol synthesis and cholesterol absorption marker levels. Patients on high potency statins had the lowest levels of cholesterol synthesis markers and the highest levels of cholesterol absorption markers at baseline, and the greatest reduction in absorption markers and the smallest increases in synthesis markers with ezetimibe addition. Compared with baseline values, ezetimibe was most effective in reducing LDL-C when added to high potency statin therapy; however, this finding was no longer significant after adjusting for placebo effects. Nevertheless, these results highlight the complementary effects that statins and ezetimibe have on modulating markers of cholesterol synthesis and absorption, and suggest that patients on high potency statins may be good candidates for ezetimibe therapy if additional LDL-C lowering is required to reach LDL-C goals.

1. A method for treating a patient with high LDL-C, the method comprising:
obtaining a sample from a patient;
conducting an assay on the sample to obtain a level of a cholesterol absorption marker and a level of a cholesterol production marker;
balancing the level of the cholesterol absorption marker against the level of the cholesterol production marker to determine whether the patient is a balanced producer, an over-producer, and an over-absorber of cholesterol; and
administering to the patient a drug that inhibits cholesterol absorption based on the balancing step, thereby treating the patient with high LDL-C.

2. The method according to claim 1, wherein the sample is a blood sample.

3. The method according to claim 2, wherein the marker is a sterol.

4. The method according to claim 3, wherein the sterol alcohol is campesterol or β-sitosterol.

5. The method according to claim 1, wherein the patient is also taking a statin.

6. The method according to claim 5, wherein the statin is a high-potency statin.

7. The method according to claim 6, wherein the high-potency statin is simvastatin or atorvastatin.

8. The method according to claim 1, wherein the drug administered is ezetimibe.

9. The method according to claim 1, wherein the balancing step comprises
comparing the levels of the cholesterol absorption and production markers to respective reference levels;
assigning values to the cholesterol absorption and production markers based on the comparison; and
balancing the value of the cholesterol absorption marker against the value of the cholesterol production marker, wherein the patient should be administered a drug that inhibits cholesterol absorption if the value of the cholesterol absorption marker exceeds the value of the cholesterol production marker.

10. The method of claim 1, wherein the patient should be administered a drug that inhibits cholesterol absorption if the patient is a balanced producer or an over-absorber.
